(19) United States
(12) Patent Application Publication
(10) Pub. No.: US 2005/0203044 A1
(43) Pub. Date: Sep. 15, 2005

(54) Small-mer compositions and methods of use

(75) Inventor: Shawn Zinnen, Denver, CO (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Sirna Therapeutics, Inc., Boulder, CO (US)

(21) Appl. No.: 11/043,673

(22) Filed: Jan. 26, 2005

Related U.S. Application Data
(63) Continuation of application No. PCT/US03/25031, filed on Aug. 8, 2003.
(60) Provisional application No. 60/402,093, filed on Aug. 8, 2002.

(51) Int. Cl. A61K 48/00; C07H 21/02

(52) U.S. Cl. 514/44; 536/23.1

(57) ABSTRACT

The present invention concerns small-mer compositions and methods useful in a variety of applications, including use in therapeutic, diagnostic, target validation, and genomic discovery applications. Specifically, the invention relates to small-mer compositions capable of inhibiting viral replication, useful in treating diseases and conditions related to viral infection, and/or capable of inhibiting cellular proliferation, useful in treating diseases and conditions related to cellular proliferation.

This application is a Continuation of International Application No. PCT/US03/025031 filed Aug. 8, 2003, which claims the benefit of U.S. Provisional Application No. 60/402,093, filed Aug. 8, 2002, incorporated by reference herein in its entirety including the drawings.

### BACKGROUND OF THE INVENTION

### Field of the Invention

The following is a discussion of relevant art pertaining to short oligonucleotides. The discussion is provided only for understanding of the invention that follows. The summary is not an admission that any of the work described below is prior art to the claimed invention.

Short oligonucleotides have potential as therapeutic agents based upon several observations. The role of nucleotides in the regulation of numerous biological processes, and their pathological conterparts, has lead to the development of nucleoside analogs as antiviral and anti-cancer agents. Adenine dinucleotides are a known set of signalling molecules with divergent biological effects. Analogs of adenine dinucleotides have been shown to be potent enzyme inhibitors as well as suppressors of normal and malignant cell proliferation (Zatorski et al., 1995, J Med. Chem., 38, 1098-1105). Thymidine dinucleotides have been shown to inhibit contact hypersensitivity and activate tumor necrosis factor alpha 1 (Cruz et al., 2000, The Journal of Investigative Dermatology, 114, 253-258). The dinucleotide 5′-Cytosine-Guanosine-3′ with a phosphorothioate internucleotide linkage is known to cause a non-specific inflammatory response, whereas CG in the context of the hexamer sequences such as NACGTN, when delivered in liposomes, induces interferon and activates natural killer cells, thus exhibiting tumor regression activity (Sonehara et al., 1996, J Interferon Cytokine Res., 16, 799-803). An investigation of all possible dinucleotides effecting HIV Integrase activity revealed three potent inhibitors (pAC, pAT, and pCT), (Mazumder et al., 1997, Molecular Pharmacology, 51, 567-575). These three dinucleotide constructs did not demonstrate antiviral activity, even though they were able to inhibit the integrase enzyme process.

There are currently few reports of any small-mers (e.g. having three or more nucleotide or non-nucleotide moieties) of less than eighteen residues in length having therpeutic activity. An anti-HIV octamer was identified by the combinatorial selection stratagy known as SURF (synthetic unrandomization of randomized fragmants), (Ecker et al., 1993, Nucleic Acids Research, 21, 1853-56). A phosphorothioate oligonucleotide octamer that forms a tetrameric guanosine-quartet structure which binds the HIV envelope protein gp120, and inhibits cell-to-cell and virus-to-cell infection with an EC of approximately 0.3-6.4 uM has been reported (Wyatt et al., 1994, PNAS USA, 91, 1356-60). This oligonucleotide has demonstrated in vivo efficacy in a SCID-hu Thy/Liv Mouse HIV model and has been reported to have toxicology profiles similar to other phosphorothioate oligonucleotides (Stoddart et al., 1998, Antimicrobial Agents and Chemotherapy, 42, 2113-15). A 17-mer oligonucleotide with two phosphorothioate internucleotide linkages has been reported to have anti-HIV activity (Bishop et al., 1996, J Biol. Chem., 271, 5698-03). This viral inhibition is proposed to be through the interaction with the HIV viral integrase, however, the similar GT content can suggest a mechanism similar to the T₂G₄T₂ octamer described above. A 15-mer thiophosphoramidate oligonucleotide designed to interact with the telomerase RNA subunit has been shown to inhibit telomerase activity (Pruzan et al., 2002, Nucleic Acids Research, 30, 559-568). Similarly, a 13-mer thiophosphoramidate oligonucleotide designed to interact with the telomerase RNA subunit has been shown to inhibit telomerase activity as well (Herbert et al., 2002, Oncogene, 21, '638-42). A phenoxazine-substituted phosphorothioate oligonucleotide targeting SV40 large T antigen has been described to have improved cellular penetration and enhanced target RNA binding properties compared to a 7-mer C-5 propynyl phosphorothioate oligonucleotide (Flanagan et al., 1999, Nature Biotechnology, 17, 48-52).

Applicant has applied a high-throughput screening approach to identify small-mers having antiviral and antiproliferative properties. The utility of the method described herein capitalizes on covering all potential sequence space for a small-mer of predetermined length (i.e., examining all combinations of nucleotides for a particular given length) to identify potent inhibitors of viral replication and/or cellular proliferation that are non-toxic to normal cells. The use of small-mer therapeutics of the invention represents a novel approach to treating diseases and conditions related to viral replication and cellular proliferation.

Kao et al., International PCT Publication No. WO 00/04141, describes linear single stranded nucleic acid molecules capable of specifically binding to viral polymerases and inhibiting the activity of the viral polymerase.

### SUMMARY OF THE INVENTION

The present invention relates to compounds, compositions, and methods useful for inhibiting viral replication and/or cellular proliferation using small-mers. In particular, the instant invention features a small-mer having about 3 to about 6 nucleotides and having antiviral or antiproliferative activity or both. The small-mer constructs of the invention can be further optimized to comprise one or more (e.g. about 1, 2, 3, 4, 5, 6 or more) additional nucleotides or non-nucleotides or both to the extent that these nucleotides or non-nucleotides do not significantly decrease the effectiveness of the small-mer construct. The small-mer constructs of the invention can be further optimized to comprise one or more (e.g. about 1, 2, 3, 4, 5, 6 or more) fewer nucleotides or non-nucleotides or both to the extent that these nucleotides or non-nucleotides do not significantly decrease the effectiveness of the small-mer construct. The small-mers of the invention can be unmodified or chemically modified. The small-mers of the instant invention can be chemically synthesized. The instant invention features various chemically modified synthetic small-mer molecules capable of inhibiting viral activity, such as HIV-1 activity, in cells. The instant invention also features various chemically modified synthetic small-mer molecules capable of inhibiting cellular proliferation. The use of chemically modified small-mers is expected to improve various properties of native small-mer molecules through increased resistance to nuclease degradation and/or improved cellular uptake in vivo and in vitro. The small-mer molecules of the instant invention provide useful reagents and methods for a variety of therapeutic, diagnostic, agricultural, target validation, genomic discovery, genetic engineering and pharmacogenomic applications.

In one embodiment, a small-mer of the invention comprises a sequence having any of SEQ ID NOs. 1-182. In another embodiment, the small-mer of the invention comprises one or more 2′-O-alkyl nucleotides, such as 2′-O-allyl nucleotides. In another example, a small-mer of the invention comprises one or more phosphorothioate internucleotide linkages. In yet another embodiment, a small-mer of the invention comprises a terminal cap moeity at the 3′-end, 5′-end, or both 3′ and 5′ ends of the small-mer molecule.

In one embodiment, a small-mer of the invention comprises one or more (e.g., 1, 2, 3, 4, 5, 6, or more) 2′-O-allyl modified nucleotides.

In one embodiment, the small-mer of the invention comprises about 3 to about 6 nucleotides in length, for example, about 3, 4, 5, or 6 nucleotides in length. In another embodiment, additional nucleotides or non-nucleotides or both can be added to or substituted or both in a small-mer of the invention, for example, about 1 to about 10 additional nucleotides or non-nucleotides or both can be added to the length of the small-mer (e.g. about 1, 2, 3, 4, 5, 6, 7, 8, 9, or 10 additional nucleotides or non-nucleotides or both) to the extent that the specificity or activity of the small-mer is not decreased, for example, where the specificity or activity or the small-mer is increased.

In one embodiment, the invention features one or more small-mer molecules and methods that independently or in combination inhibit viral replication or infection or both. Specifically, the present invention features small-mer molecules with antiviral activity against a virus, for example, viruses including but not limited to Hepatitis C Virus (HCV, for example Genbank Accession Nos: D11168, D50483.1, L38318 and S82227), Hepatitis B Virus (HBV, for example GenBank Accession No. AF100308.1), Human Immunodeficiency Virus type 1 (HIV-1, for example GenBank Accession No. U51188), Human Immunodeficiency Virus type 2 (HIV-2, for example GenBank Accession No. X60667), West Nile Virus (WNV for example GenBank accession No. NC_{—}001563), cytomegalovirus (CMV for example GenBank Accession No. NC_{—}001347), respiratory syncytial virus (RSV for example GenBank Accession No. NC_{—}001781), influenza virus (for example example GenBank Accession No. AF037412, rhinovirus (for example, GenBank accession numbers: D00239, X02316, X01087, L24917, M16248, K02121, X01087), papillomavirus (for example GenBank Accession No. NC_{—}001353), Herpes Simplex Virus (HSV for example GenBank Accession No. NC_{—}001345), and other viruses such as HTLV (for example GenBank Accession No. AJ430458).

In one embodiment, the invention features chemically modified small-mer constructs having antiviral or antiproliferative activity or both. Non-limiting examples of such chemical modifications include without limitation phosphorothioate internucleotide linkages, 2′-O-allyl ribonucleotides, 2′-O-methyl ribonucleotides, 2′-deoxy-2′-fluoro ribonucleotides, “universal base” nucleotides, locked nucleic acid (LNA) nucleotides, and inverted deoxyabasic residue incorporation.

In a non-limiting example, the introduction of chemically modified nucleotides into nucleic acid molecules of the invention will provide a powerful tool in overcoming potential limitations of in vivo stability and bioavailability inherent to native RNA molecules that are delivered exogenously. For example, the use of chemically modified nucleic acid molecules can enable a lower dose of a particular nucleic acid molecule for a given therapeutic effect since chemically modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically modified nucleic acid molecule is reduced as compared to a native nucleic acid molecule, for example when compared to an all RNA nucleic acid molecule, the overall activity of the modified nucleic acid molecule can be greater than the native molecule due to improved stability and/or delivery of the molecule.

In one embodiment, the invention features a chemically modified small-mer molecule having antiviral or antiproliferative activity or both, wherein the chemical modification comprises one or more nucleotides comprising a backbone modified internucleotide linkage having Formula I: 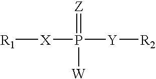
wherein each R1 and R2 is independently any nucleotide, non-nucleotide, or small-mer which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all O. In another embodiment, a small-mer molecule of the invention having internucleotide linkage(s) of Formula I also comprises a chemically modified nucleotide or non-nucleotide having any of Formulae II, III, V, or VI.

In one embodiment, the invention features a chemically modified small-mer molecule, wherein the chemical modification comprises one or more nucleotides or non-nucleotides having Formula II: 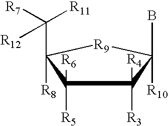
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base or a non-nucleosidic base such as phenyl, naphthyl, 3-nitropyrrole, 5-nitroindole, nebularine, pyridone, pyridinone, or any other non-naturally occurring universal base.

In one embodiment, the invention features a chemically modified small-mer molecule, wherein the chemical modification comprises one or more nucleotides or non-nucleotides having Formula III: 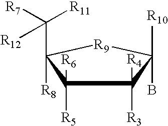
wherein each R3, R4, R5, R6, R7, R8, R10, R11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base such as adenine, guanine, uracil, cytosine, thymine, 2-aminoadenosine, 5-methylcytosine, 2,6-diaminopurine, or any other non-naturally occurring base or a non-nucleosidic base such as phenyl, naphthyl, 3-nitropyrrole, 5-nitroindole, nebularine, pyridone, pyridinone, or any other non-naturally occurring universal base.

In another embodiment, a small-mer molecule of the invention comprises a nucleotide having Formula II or III, wherein the nucleotide having Formula II or III is in an inverted configuration. For example, the nucleotide having Formula II or III is connected to the small-mer construct in a 3′,3′, 3′-2′,2′-3′, or 5′,5′ configuration, such as at the 3′-end, 5′-end, or both 3′ and 5′ ends of one or both small-mer strands.

In one embodiment, the invention features a chemically modified small-mer molecule, wherein the chemical modification comprises a 5′-terminal phosphate group having Forula IV: 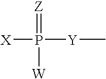
wherein each X and Y is independently O, S, N, alkyl, substituted alkyl, or alkylhalo; each Z and W is independently O, S, N, alkyl, substituted alkyl, Q-alkyl, S-alkyl, alkaryl, aralkyl, or alkylhalo.

In one embodiment, the invention features a chemically modified small-mer molecule, wherein the chemical modification comprises one or more phosphorothioate internucleotide linkages. For example, in a non-limiting example, the invention features a chemically modified small-mer having about 1, 2, 3, 4, 5, 6 or more phosphorothioate internucleotide linkages. In another non-limiting example, an exemplary small-mer molecule of the invention can comprise one or more (e.g., about 1, 2, 3, 4, 5, 6 or more) pyrimidine phosphorothioate internucleotide linkages. In yet another non-limiting example, an exemplary small-mer molecule of the invention can comprise one or more (e.g., about 1, 2, 3, 4, 5, 6 or more) purine phosphorothioate internucleotide linkages.

In another embodiment, the invention features a small-mer molecule comprising one or more 2′-5′ internucleotide linkages, for example about 1, 2, 3, 4, 5, 6 or more 2′-5′ internucleotide linkages.

In one embodiment, a small-mer molecule of the invention comprises one or more abasic residues, for example a compound having Formula V: 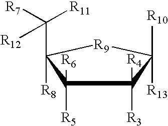
wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2.

In one embodiment, a small-mer molecule of the invention comprises one or more inverted abasic residues, for example a compound having Formula VI: 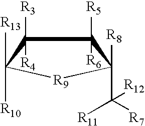
wherein each R3, R4, R5, R6, R7, R8, R10, R11, R12, and R13 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO2, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and either R2, R3, R8 or R13 serve as points of attachment to the small-mer molecule of the invention.

In another embodiment, a small-mer molecule of the invention comprises an abasic residue having Formula II or III, wherein the abasic residue having Formula II or III is connected to the small-mer in a 3′, 3′, 3′-2′,2′-3′, or 5′, 5′ configuration, such as at the 3′-end, 5′-end, or both 3′ and 5′ ends of the small-mer.

In one embodiment, a small-mer molecule of the invention comprises one or more locked nucleic acid (LNA) nucleotides, for example at the 5′-end, 3′-end, 5′ and 3′-end, or any combination thereof of the small-mer molecule.

In another embodiment, a small-mer molecule of the invention comprises one or more acyclic nucleotides, for example at the 5′-end, 3′-end, 5′ and 3′-end, or any combination thereof, of the small-mer molecule.

In one embodiment, the invention features a chemically modified small-mer molecule, wherein the chemical modification comprises a conjugate covalently attached to the small-mer molecule. In another embodiment, the conjugate is covalently attached to the small-mer molecule via a biodegradable linker. In one embodiment, the conjugate molecule is attached at the 3′-end of the small-mer. In another embodiment, the conjugate molecule is attached at the 5′-end of the small-mer. In yet another embodiment, the conjugate molecule is attached at both the 3′-end and 5′-end of the small-mer. In one embodiment, a conjugate molecule of the invention comprises a molecule that facilitates delivery of a small-mer molecule into a biological system such as a cell. In another embodiment, the conjugate molecule attached to the small-mer is a polyethylene glycol, human serum albumin, or a ligand for a cellular receptor that can mediate cellular uptake. Examples of specific conjugate molecules contemplated by the instant invention that can be attached to small-mer molecules are described in Vargeese et al., U.S. Ser. No. 10/501,394, incorporated by reference herein.

In one embodiment, the invention features a method for inhibiting viral activity or replication within a cell comprising: (a) synthesizing a small-mer molecule of the invention, which can be chemically modified; and (b) introducing the small-mer molecule into a cell under conditions suitable to inhibit viral activity or replication in the cell.

In another embodiment, the invention features a method for inhibiting cellular proliferation comprising: (a) synthesizing a small-mer molecule of the invention, which can be chemically modified; and (b) introducing the small-mer molecule into a cell under conditions suitable to inhibit proliferaction of the cell.

In one embodiment, the invention features a method of inhibiting viral activity or replication in a tissue explant comprising: (a) synthesizing a small-mer molecule of the invention, which can be chemically modified; (b) introducing the small-mer molecule into a cell of the tissue explant derived from a particular organism under conditions suitable to inhibit viral activity or replication in the tissue explant; and (c) optionally introducing the tissue explant back into the organism the tissue was derived from or into another organism under conditions suitable to inhibit viral activity or replication in that organism.

In one embodiment, the invention features a method of inhibiting cellular proliferation in a tissue explant comprising: (a) synthesizing a small-mer molecule of the invention, which can be chemically modified; (b) introducing the small-mer molecule into a cell of the tissue explant derived from a particular organism under conditions suitable to inhibit cellular proliferation in the tissue explant, and (c) optionally introducing the tissue explant back into the organism the tissue was derived from or into another organism under conditions suitable to inhibit viral activity or replication in that organism.

In one embodiment, the invention features a method of inhibiting viral activity or replication in an organism comprising: (a) synthesizing a small-mer molecule of the invention, which can be chemically modified; and (b) introducing the small-mer molecule into the organism under conditions suitable to inhibit viral activity or replication in the organism.

In one embodiment, the invention features a composition comprising a small-mer molecule of the invention, which can be chemically modified, in a pharmaceutically acceptable carrier or diluent. In another embodiment, the invention features a pharmaceutical composition comprising small-mer molecules of the invention, which can be chemically modified, targeting one or more viruses or cell types, in a pharmaceutically acceptable carrier or diluent. In another embodiment, the invention features a method for treating or preventing a disease or condition in a subject comprising administering to the subject a composition of the invention under conditions suitable for the treatment or prevention of the disease or condition in the subject, alone or in conjunction with one or more other therapeutic compounds. In yet another embodiment, the invention features a method for reducing or preventing tissue rejection in a subject comprising administering to the subject a composition of the invention under conditions suitable for the reduction or prevention of tissue rejection in the subject.

In one embodiment, the invention features a kit containing a small-mer molecule of the invention, which can be chemically modified, that can be used to inhibit viral activity or replication or both in a cell, tissue, or organism. In another embodiment, the invention features a kit containing more than one (e.g. about 1, 2, 3, 4, 5, 6, 7, 8, 9, 10 or more) small-mer molecule of the invention, which can be chemically modified, that can be used to inhibit viral activity or replication or both in a cell, tissue, or organism.

In another embodiment, the invention features a kit containing a small-mer molecule of the invention, which can be chemically modified, that can be used to inhibit cellular proliferation in a tissue or organism. In another embodiment, the invention features a kit containing more than one (e.g. about 1, 2, 3, 4, 5, 6, 7, 8, 9, 10 or more) small-mer molecule of the invention, which can be chemically modified, that can be used to inhibit cellular proliferation in a tissue, or organism.

In one embodiment, the invention features a cell containing one or more small-mer molecules of the invention, which can be chemically modified. In another embodiment, the cell containing a small-mer molecule of the invention is a mammalian cell. In yet another embodiment, the cell containing a small-mer molecule of the invention is a human cell.

In one embodiment, the invention features a small-mer, wherein the small-mer comprises one or more chemical modifications, for example one or more chemical modifications having Formula I, II, III, IV, or V, that increases the nuclease resistance of the small-mer.

In another embodiment, the invention features a method for generating small-mer molecules with increased nuclease resistance comprising (a) introducing nucleotides or non-nucleotides having any of Formula I-VI into a small-mer molecule, and (b) assaying the small-mer molecule of step (a) under conditions suitable for isolating small-mer molecules having increased nuclease resistance.

In another embodiment, the invention features a method for generating small-mer molecules with improved antiviral activity comprising (a) introducing nucleotides or non-nucleotides having any of Formula I-VI into a small-mer molecule, and (b) assaying the small-mer molecule of step (a) under conditions suitable for isolating small-mer molecules having improved antiviral activity.

In another embodiment, the invention features a method for generating small-mer molecules with improved antiproliferative activity comprising (a) introducing nucleotides or non-nucleotides having any of Formula I-VI into a small-mer molecule, and (b) assaying the small-mer molecule of step (a) under conditions suitable for isolating small-mer molecules having improved antiproliferative activity.

In one embodiment, the invention features a small-mer molecule, wherein the small-mer comprises one or more chemical modifications described herein that modulates the cellular uptake of the small-mer.

In another embodiment, the invention features a method for generating small-mer molecules with improved cellular uptake comprising (a) introducing nucleotides or non-nucleotides having any of Formula I-VI into a small-mer molecule, and (b) assaying the small-mer molecule of step (a) under conditions suitable for isolating small-mer molecules having improved cellular uptake.

In one embodiment, the invention features small-mer molecules with antiviral or antiproliferative activity or both, wherein the small-mer comprises one or more (e.g. about 1, 2, 3, 4, 5, 6, 7, 8, 9, 10 or more) chemical modifications described herein that increases the bioavailability of the small-mer, for example, by attaching polymeric conjugates such as polyethyleneglycol or equivalent conjugates that improve the pharmacokinetics of the small-mer, or by attaching conjugates that target specific tissue types or cell types in vivo. Non-limiting examples of such conjugates are described in Vargeese et al., U.S. Ser. No. 10/201,394 incorporated by reference herein.

In one embodiment, the invention features a method for generating small-mer molecules of the invention with improved bioavailability comprising (a) introducing a conjugate into the structure of a small-mer molecule, and (b) assaying the small-mer molecule of step (a) under conditions suitable for isolating small-mer molecules having improved bioavailability. Such conjugates can include ligands for cellular receptors such as peptides derived from naturally occurring protein ligands, protein localization sequences including cellular ZIP code sequences, antibodies, nucleic acid aptamers, vitamins and other co-factors such as folate and N-acetylgalactosamine, polymers such as polyethyleneglycol (PEG), phospholipids, polyamines such as spermine or spermidine, and others.

In another embodiment, the invention features a method for generating small-mer molecules of the invention with improved bioavailability comprising (a) introducing an excipient formulation to a small-mer molecule, and (b) assaying the small-mer molecule of step (a) under conditions suitable for isolating small-mer molecules having improved bioavailability. Such excipients include polymers such as cyclodextrins, lipids, cationic lipids, polyamines, phospholipids, and others.

In another embodiment, the invention features a method for generating small-mer molecules of the invention with improved bioavailability comprising (a) introducing nucleotides or non-nucleotides having any of Formula I-VI into a small-mer molecule, and (b) assaying the small-mer molecule of step (a) under conditions suitable for isolating small-mer molecules having improved bioavailability.

In another embodiment, polyethylene glycol (PEG) can be covalently attached to small-mer compounds of the present invention. The attached PEG can be any molecular weight, preferably from about 2,000 to about 50,000 daltons (Da).

In one embodiment, the invention features a method for generating small-mer molecules with antiviral activity comprising (a) generating a library of all possible nucleotide or non-nucleotide or both combinations for a fixed small-mer length, and (b) assaying the small-mer molecule of step (a) under conditions suitable for isolating small-mer molecules having antiviral activity. The library can comprise small-mers having one or more chemical modifications described herein, for example, in fixed or variable positions of the small-mer. The fixed small-mer length can be about 3 to about 6 or more nucleotides in length, for example 2, 3, 4, 5, 6, 7, 8, 9, 10 or more residues in length.

In another embodiment, the invention features a method for generating small-mer molecules with improved antiviral activity comprising (a) providing a small-mer of the invention having antiviral activity as a scaffold for additional nucleotides or non-nucleotides or both, (b) generating a library of small-mers by extending the length of the small-mer scaffold using all possible nucleotide or non-nucleotide or both combinations for a fixed additional small-mer length, and (c) assaying the small-mer molecule of step (b) under conditions suitable for isolating small-mer molecules having improved antiviral activity. In another embodiment, the fixed additional small-mer length is about 1 to about 10 more nucleotides in length, for example 1, 2, 3, 4, 5, 6, 7, 8, 9, 10, or more nucleotides in length, which can be added at the 3′-end, 5′-end, or both 3′ and 5′ ends of the scaffold sequence.

In one embodiment, the invention features a method for generating small-mer molecules with antiproliferative activity comprising (a) generating a library of all possible nucleotide or non-nucleotide or both combinations for a fixed small-mer length, and (b) assaying the small-mer molecule of step (a) under conditions suitable for isolating small-mer molecules having antiproliferative activity. The library can comprise small-mers having one or more chemical modifications described herein, for example in fixed or variable positions of the small-mer. In another embodiment, the fixed small-mer length is about 1 to about 10 or more nucleotides in length, for example about 1, 2, 3, 4, 5, 6, 7, 8, 9, 10, or more nucleotides in length.

In another embodiment, the invention features a method for generating small-mer molecules with improved antiproliferative activity comprising (a) providing a small-mer of the invention having antiproliferative activity as a scaffold for additional nucleotides and/or non-nucleotides, (b) generating a library of small-mers by extending the length of the small-mer scaffold using all possible nucleotide and/or non-nucleotide combinations for a fixed additional small-mer length, and (c) assaying the small-mer molecule of step (b) under conditions suitable for isolating small-mer molecules having improved antiproliferative activity. In another embodiment, the fixed additional small-mer length is about 1 to about 10 or more nucleotides in length, for example, about 1, 2, 3, 4, 5, 6, 7, 8, 9, 10, or more nucleotides in length.

In one embodiment, a small-mer molecule of the invention having antiviral activity against HIV-1 comprises a compound having Formula VII:
(N)X—G—G—G—(N)ₓ
wherein G represents any Guanosine nucleotide which can be unmodified or chemically modified as described herein, such as with 2′-O-alkyl modifications; N stands for any nucleotide or non-nucleotide; and wherein X is any integer from about 0 to about 5, for example about 0, 1, 2, 3, 4, or 5.

In one embodiment, the small-mer molecules of the invention are chemically synthesized on a high-throughput multiwell or multiplate solid phase synthesis format as described herein.

The term “small-mer” as used herein refers to a single stranded nucleic acid molecule having about 3 to about 6 nucleotides or non-nucleotides or both, for example, about 3, 4, 5, or 6 nucleotides or non-nucleotides in length. The nucleotides and non-nucleotides can be naturally occurring or chemically modified as described herein. Additional nucleotides or non-nucleotides or both can be added to a small-mer of the invention, for example, about 1 to about 10 additional nucleotides or non-nucleotides can be added, (eg. about 1, 2, 3, 4, 5, 6, 7, 8, 9, or 10 additional nucleotides or non-nucleotides) to the extent that the specificity or activity of the small-mer is not decreased, for example, where the specificity or activity or the small-mer is increased.

The term “fixed small-mer length” as used herein refers to a predetermined number of nucleotides, non-nucleotides or both present in the small-mer, for example, about 3 to 6 (e.g., about 3, 4, 5, or 6) nucleotides, non-nucleotides or both. The fixed small-mer length can be used to probe or assay all possible combinations of nucleotides, non-nucleotides or both within the small-mer sequence.

The term “antiviral” as used herein refers to the reduction of the activity, infectivity, replication or combination thereof of a virus, for example, in the presence of a small-mer of the invention below a level observed in the absense of the small-mer of the invention.

The term “antiproliferative” as used herein refers to the reduction of proliferation of a cell, for example, in the presence of a small-mer of the invention below a level observed in the absense of the small-mer of the invention.

The small-mer molecules of the invention represent a novel therapeutic approach to treat a variety of pathologic indications or other conditions, such as cancers and viral infection and any other diseases or conditions that are related to or will respond to the level of virus in a cell or tissue or the proliferaction of cells, alone or in combination with other therapies. The reduction of virus or cellular proliferaction or both relieves, to some extent, the symptoms of the disease or condition.

In one embodiment, a small-mer of the invention comprises about 3 to about 6 nucleotides or non-nucleotides, for example is about 3, 4, 5, or 6 nucleotides or non-nucleotides in length. In another embodiment, additional nucleotides or non-nucleotides or both can be added to the small-mer of the invention, for example about 1 to about 10 additional nucleotides or non-nucleotides can be added, (e.g. about 1, 2, 3, 4, 5, 6, 7, 8, 9, or 10 additional nucleotides or non-nucleotides) such that the specificity or activity of the small-mer is not decreased, for example, where the specificity or activity or the small-mer is increased. Exemplary small-mer molecules of the invention are shown in Table I (all sequences are shown 5′-3′).

As used herein, the term “cell” is used in its usual biological sense, and does not refer to an entire multicellular organism, e.g., specifically does not refer to a human. The cell can be present in an organism, e.g., mammals such as humans, cows, sheep, apes, monkeys, swine, dogs, and cats. The cell can be eukaryotic (e.g., a mammalian cell, such as a human cell). The cell can be of somatic or germ line origin, totipotent or pluripotent, dividing or non-dividing. The cell can also be derived from or can comprise a gamete or embryo, a stem cell, or a fully differentiated cell.

The small-mer molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers. In particular embodiments, the nucleic acid molecules of the invention comprise sequences shown in Table I. Examples of such nucleic acid molecules consist essentially of sequences defined in these tables/figures.

In another aspect, the invention provides mammalian cells containing one or more small-mer molecules of this invention.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a subject is a human or human cells.

The term “phosphorothioate” as used herein refers to an internucleotide linkage having Formula I, wherein Z or W or both comprise a sulfur atom. Hence, the term phosphorothioate refers to both phosphorothioate and phosphorodithioate internucleotide linkages.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example Loakes, 2001, Nucleic Acids Research, 29, 2437-2447).

The term “acyclic nucleotide” as used herein refers to any nucleotide having an acyclic ribose sugar, for example, where any of the ribose carbons (C1, C2, C3, C4, or C5), are independently or in combination absent from the nucleotide.

The nucleic acid molecules of the instant invention, individually, or in combination or in conjunction with other drugs, can be used to treat diseases or conditions discussed herein. For example, to treat a particular disease or condition, the small-mer molecules can be administered to a subject or can be administered to other appropriate cells evident to those skilled in the art, individually or in combination with one or more drugs under conditions suitable for the treatment.

In a further embodiment, the small-mer molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a small-mer molecule of the invention are enzymatic nucleic acid molecules, allosteric nucleic acid molecules, siRNA, antisense, decoy, or aptamer nucleic acid molecules, antibodies such as monoclonal antibodies, small molecules, nucleotide analogs and other organic or inorganic or both compounds including metals, salts and ions.

In one embodiment, the invention features an expression vector comprising a nucleic acid sequence encoding at least one small-mer molecule of the invention, in a manner which allows expression of the small-mer molecule.

In yet another embodiment, the expression vector of the invention comprises a sequence for a small-mer molecule having any of SEQ ID NOs. 1-182.

In one embodiment, an expression vector of the invention comprises a nucleic acid sequence encoding two or more small-mer molecules, which can be the same or different.

Other features and advantages of the invention will be apparent from the following description of the preferred embodiments thereof, and from the claims.

### BRIEF DESCRIPTION OF THE DRAWINGS

FIG. 1 shows a non-limiting example of a scheme for the synthesis of small-mer molecules of the invention. All possible combinations of a fixed sequence space are synthesized via high throughput solid phase synthesis and are then tested for antiviral and/or antiproliferative activity.

FIG. 2 shows a non-limiting example of a scheme for the combinatorial extension of an active small-mer sequence to a potentially more active sequence. In this example, a 5-mer small-mer sequence demonstrating 33% reduction of proliferation in MCF-7 cells is extended in length by 1, 2, or 3 nucleotides at the 3′ or 5′-end of the small-mer via a combinatorial approach in which all possible combinations of A, G, C, and U are tested in the extended sequence space, resulting in 168 sequences for screening. The addition of additional small-mers can possibly improve the antiproliferative properties of the original sequence.

FIG. 3 shows a sequence specific comparison of EC50 values of smallmer molecules of the invention against HIV infectivity as determined by CEM assay. Sequences shown in the Figure are completely 2′-O-allyl modified and further comprise a 3′-inverted deoxy abasic moiety.

FIG. 4 shows non-limiting examples of anti-HIV activity of smallmer molecules having GGG and GGGG motifs in CEM and MAGI assays. Sequences shown in the Figure are modified with 2′-O-allyl, 2′-O-methyl, or 2′-deoxy nucleotides as indicated, iB stands for 3′-inverted deoxy abasic moiety.

FIG. 5 shows a non-limiting example of a time of addition assay to determine small-mer activity in an anti-HIV MAGI assay.

FIG. 6 shows a non-limiting example of a time of addition assay to determine small-mer activity in an anti-HIV MAGI assay.

FIG. 7 shows a non-limiting example of a time of addition assay in MAGI cells indicating that small-mer molecules of the invention inhibit viral entry into cells.

### DETAILED DESCRIPTION OF THE INVENTION

Synthesis of Nucleic acid Molecules

Oligonucleotides and small-mers (e.g., certain modified oligonucleotides or portions of oligonucleotides lacking ribonucleotides) are synthesized using protocols known in the art, for example as described in Caruthers et al., 1992, Methods in Enzymology 211, 3-19, Thompson et al., International PCT Publication No. WO 99/54459, Wincott et al., 1995, Nucleic Acids Res. 23, 2677-2684, Wincott et al., 1997, Methods Mol. Bio., 74, 59, Brennan et al., 1998, Biotechnol Bioeng., 61, 33-45, and Brennan, U.S. Pat. No. 6,001,311. All of these references are incorporated herein by reference. The synthesis of small-mers makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 2.5 min coupling step for 2′-O-methylated nucleotides and a 45 second coupling step for 2′-deoxy nucleotides or 2′-deoxy-2′-fluoro nucleotides. Table IV outlines the amounts and the contact times of the reagents used in the synthesis cycle. Alternatively, syntheses at the 0.2 μmol scale can be performed on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 105-fold excess of S-ethyl tetrazole (60 μL of 0.25 M=15 μmol) can be used in each coupling cycle of 2′-O-methyl nucleotides relative to polymer-bound 5′-hydroxyl. A 22-fold excess (40 μL of 0.11 M=4.4 μmol) of deoxy phosphoramidite and a 70-fold excess of S-ethyl tetrazole (40 μL of 0.25 M=10 μmol) can be used in each coupling cycle of deoxy nucleotides relative to polymer-bound 5′-hydroxyl. Average coupling yields on the 394 Applied Biosystems, Inc. synthesizer, determined by calorimetric quantitation of the trityl fractions, are typically 97.5-99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide, 0.05 M in acetonitrile) is used.

Deprotection of the DNA-based small-mers is performed as follows: the polymer-bound trityl-on small-mer is transferred to a 4 mL glass screw top vial and suspended in a solution of 40% aq. methylamine (1 mL) at 65° C. for 10 minutes. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the small-mer, are dried to a white powder.

The method of synthesis used for RNA including certain small-mer molecules of the invention follows the procedure as described in Usman et al., 1987, J. Am. Chem. Soc., 109, 7845; Scaringe et al., 1990, Nucleic Acids Res., 18, 5433; and Wincott et al., 1995, Nucleic Acids Res. 23, 2677-2684 Wincott et al., 1997, Methods Mol. Bio., 74, 59, and makes use of common nucleic acid protecting and coupling groups, such as dimethoxytrityl at the 5′-end, and phosphoramidites at the 3′-end. In a non-limiting example, small scale syntheses are conducted on a 394 Applied Biosystems, Inc. synthesizer using a 0.2 μmol scale protocol with a 7.5 minute coupling step for alkylsilyl protected nucleotides and a 2.5 minute coupling step for 2′-O-methylated nucleotides. Table IV outlines the amounts and the contact times of the reagents used in the synthesis cycle. Alternatively, syntheses at the 0.2 μmol scale can be done on a 96-well plate synthesizer, such as the instrument produced by Protogene (Palo Alto, Calif.) with minimal modification to the cycle. A 33-fold excess (60 μL of 0.11 M=6.6 μmol) of 2′-O-methyl phosphoramidite and a 75-fold excess of S-ethyl tetrazole (60 μL of 0.25 M=15 μmol) can be used in each coupling cycle of 2′-O-methyl nucleotides relative to polymer-bound 5′-hydroxyl. A 66-fold excess (120 μL of 0.11 M=13.2 μmol) of alkylsilyl (ribo) protected phosphoramidite and a 150-fold excess of S-ethyl tetrazole (120 μL of 0.25 M=30 μmol) can be used in each coupling cycle of ribo nucleotides relative to polymer-bound 5′-hydroxyl. Average coupling yields on the 394 Applied Biosystems, Inc. synthesizer, determined by colorimetric quantitation of the trityl fractions, are typically 97.5 99%. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); oxidation solution is 16.9 mM 12, 49 mM pyridine, 9% water in THF (PERSEPTIVE™). Burdick & Jackson Synthesis Grade acetonitrile is used directly from the reagent bottle. S-Ethyltetrazole solution (0.25 M in acetonitrile) is made up from the solid obtained from American International Chemical, Inc. Alternately, for the introduction of phosphorothioate linkages, Beaucage reagent (3H-1,2-Benzodithiol-3-one 1,1-dioxide 0.05 M in acetonitrile) is used.

Deprotection of the RNA is performed using either a two-pot or one-pot protocol. For the two-pot protocol, the polymer-bound trityl-on small-mer is transferred to a 4 mL glass screw top vial and suspended in a solution of 40% aq. methylamine (1 mL) at 65° C. for 10 minutes. After cooling to −20° C., the supernatant is removed from the polymer support. The support is washed three times with 1.0 mL of EtOH:MeCN:H₂O/3:1:1, vortexed and the supernatant is then added to the first supernatant. The combined supernatants, containing the small-mer, are dried to a white powder. The base deprotected small-mer is resuspended in anhydrous TEA/HF/NMP solution (300 μL of a solution of 1.5 mL N-methylpyrrolidinone, 750 μL TEA and 1 mL TEA·3HF to provide a 1.4 M HF concentration) and heated to 65° C. After 1.5 hour, the small-mer is quenched with 1.5 M NH₄HCO₃.

Alternatively, for the one-pot protocol, the polymer-bound trityl-on small-mer is transferred to a 4 mL glass screw top vial and suspended in a solution of 33% ethanolic methylamine/DMSO: 1/1 (0.8 mL) at 65° C. for 15 minutes. The vial is brought to r.t. TEA·3HF (0.1 mL) is added and the vial is heated at 65° C. for 15 minutes. The sample is cooled at −20° C. and then quenched with 1.5 M NH₄HCO₃.

For purification of the trityl-on small-mer, the quenched NH₄HCO₃ solution is loaded onto a C-18 containing cartridge that had been prewashed with acetonitrile followed by 50 mM TEAA. After washing the loaded cartridge with water, the small-mer is detritylated with 0.5% TFA for 13 minutes. The cartridge is then washed again with water, salt exchanged with 1 M NaCl and washed with water again. The small-mer is then eluted with 30% acetonitrile.

The average stepwise coupling yields are typically >98% (Wincott et al., 1995 Nucleic Acids Res. 23, 2677-2684). Those of ordinary skill in the art will recognize that the scale of synthesis can be adapted to be larger or smaller than the example described above including but not limited to 96-well format, all that is important is the ratio of chemicals used in the reaction.

Alternatively, the nucleic acid molecules of the present invention can be synthesized separately and joined together post-synthetically, for example, by ligation (Moore et al., 1992, Science 256, 9923; Draper et al., International PCT publication No. WO 93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis or deprotection or both.

The nucleic acid molecules of the present invention can be modified extensively to enhance stability by modification with nuclease resistant groups, for example, 2′-O-allyl, 2′-amino, 2′-C-allyl, 2′-flouro, 2′-O-methyl, 2′-H (for a review see Usman and Cedergren, 1992, TIBS 17, 34; Usman et al., 1994, Nucleic Acids Symp. Ser. 31, 163). Small-mer constructs can be purified by gel electrophoresis using general methods or can be purified by high pressure liquid chromatography (HPLC; see Wincott et al., supra, the totality of which is hereby incorporated herein by reference) and re-suspended in water.

Optimizing Activity of the Small-Mers of the Invention.

Chemically synthesizing nucleic acid molecules with modifications (base, sugar and/or phosphate) can prevent their degradation by serum ribonucleases, which can increase their potency (see e.g., Eckstein et al., International Publication No. WO 92/07065; Perrault et al., 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; Gold et al., U.S. Pat. No. 6,300,074; and Burgin et al., supra; all of which are incorporated by reference herein). All of the above references describe various chemical modifications that can be made to the base, phosphate or sugar moieties or both of the nucleic acid molecules described herein. Modifications that enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides are modified to enhance stability or enhance biological activity or both by modification with nuclease resistant groups, for example, 2′-amino, 2′-C-allyl, 2′-flouro, 2′-O-methyl, 2′-O-allyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565-568; Pieken et al. Science, 1991, 253, 314-317; Usman and Cedergren, Trends in Biochem. Sci., 1992, 17, 334-339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., U.S. Pat. No. 5,627,053; Woolf et al., International PCT Publication No. WO 98/13526; Thompson et al., U.S. Ser. No. 60/082,404 which was filed on Apr. 20, 1998; Karpeisky et al., 1998, Tetrahedron Lett., 39, 1131; Eamshaw and Gait, 1998, Biopolymers (Nucleic Acid Sciences), 48, 39-55; Verma and Eckstein, 1998, Annu. Rev. Biochem., 67, 99-134; and Burlina et al., 1997, Bioorg. Med. Chem., 5, 1999-2010; all of the references are hereby incorporated in their totality by reference herein). Such publications describe general methods and strategies to determine the location of incorporation of sugar, base or phosphate or a combination thereof modifications and the like into nucleic acid molecules without modulating catalysis, and are incorporated by reference herein. In view of such teachings, similar modifications can be used as described herein to modify the small-mer nucleic acid molecules of the instant invention so long as the antiviral and/or antiproliferative activity of the small-mer cells is not significantly inhibited.

Small-mer molecules having chemical modifications that maintain or enhance activity are provided. Such a nucleic acid is also generally more resistant to nucleases than an unmodified nucleic acid.

In one embodiment, small-mer molecules of the invention include one or more G-clamp nucleotides. A G-clamp nucleotide is a modified cytosine analog wherein the modifications confer the ability to hydrogen bond both Watson-Crick and Hoogsteen faces of a complementary guanine within a duplex, see for example Lin and Matteucci, 1998, J. Am. Chem. Soc., 120, 8531-8532. A single G-clamp analog substitution within an oligonucleotide can result in substantially enhanced helical thermal stability and mismatch discrimination when hybridized to complementary oligonucleotides. The inclusion of such nucleotides in nucleic acid molecules of the invention results in both enhanced affinity and specificity to nucleic acid targets, complementary sequences, or template strands. In another embodiment, nucleic acid molecules of the invention include one or more LNA “locked nucleic acid” nucleotides such as a 2′,4′-C mythylene bicyclo nucleotide (see for example Wengel et al., International PCT Publication No. WO 00/66604 and WO 99/14226).

In another embodiment, the invention features conjugates or complexes or both of small-mer molecules of the invention. Such conjugates or complexes or both can be used to facilitate delivery of small-mer molecules into a biological system, such as a cell. The conjugates and complexes provided by the instant invention can impart therapeutic activity by transferring therapeutic compounds across cellular membranes, altering the pharmacokinetics, or modulating or both the localization of nucleic acid molecules of the invention. The present invention encompasses the design and synthesis of novel conjugates and complexes for the delivery of molecules, including, but not limited to, small molecules, lipids, phospholipids, nucleosides, nucleotides, nucleic acids, antibodies, toxins, negatively charged polymers and other polymers, for example proteins, peptides, hormones, carbohydrates, polyethylene glycols, or polyamines, across cellular membranes. In general, the transporters described are designed to be used either individually or as part of a multi-component system, with or without degradable linkers. These compounds are expected to improve delivery or localization or both of nucleic acid molecules of the invention into a number of cell types originating from different tissues, in the presence or absence of serum (see Sullenger and Cech, U.S. Pat. No. 5,854,038). Conjugates of the molecules described herein can be attached to biologically active molecules via linkers that are biodegradable, such as biodegradable nucleic acid linker molecules.

The term “biodegradable nucleic acid linker molecule” as used herein, refers to a nucleic acid molecule that is designed as a biodegradable linker to connect one molecule to another molecule, for example, a biologically active molecule. The stability of the biodegradable nucleic acid linker molecule can be modulated by using various combinations of ribonucleotides, deoxyribonucleotides, and chemically modified nucleotides, for example, 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications or a combination thereof.

The term “biodegradable” as used herein, refers to degradation in a biological system, for example, enzymatic degradation or chemical degradation.

The term “biologically active molecule” as used herein, refers to compounds or molecules that are capable of eliciting or modifying a biological response in a system. Non-limiting examples of biologically active small-mer molecules either alone or in combination with the molecules contemplated by the instant invention include therapeutically active molecules such as antibodies, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, small-mer, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

In another aspect a small-mer molecule of the invention comprises one or more 5′ or 3′-cap structure or both, for example, on only the sense small-mer strand, antisense small-mer strand, or both small-mer strands.

By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and can help in delivery or localization or both within a cell. The cap can be present at the 5′-terminus (5′-cap) or at the 3′-terminal (3′-cap) or can be present on both termini. In non-limiting examples, the 5′-cap is selected from an inverted abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In another embodiment, the 3′-cap is selected from a 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging or non-bridging or both 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

By the term “non-nucleotide” is meant any group or compound which can be incorporated into a nucleic acid chain in the place of one or more nucleotide units, including either sugar or phosphate substitutions or both, and allows the remaining bases to exhibit their activity. The group or compound is abasic in that it does not contain a commonly recognized nucleotide base, such as adenosine, guanine, cytosine, uracil or thymine and therefore lacks a base at the 1′-position.

An “alkyl” group refers to a saturated aliphatic hydrocarbon, including straight-chain, branched-chain, and cyclic alkyl groups. Preferably, the alkyl group has 1 to 12 carbons. More preferably, it is a lower alkyl of from 1 to 7 carbons, more preferably 1 to 4 carbons. The alkyl group can be substituted or unsubstituted. When substituted the substituted group(s) is preferably, hydroxyl, cyano, alkoxy, ═O, ═S, NO₂ or N(CH₃)₂, amino, or SH. The term also includes alkenyl groups that are unsaturated hydrocarbon groups containing at least one carbon-carbon double bond, including straight-chain, branched-chain, and cyclic groups. Preferably, the alkenyl group has 1 to 12 carbons. More preferably, it is a lower alkenyl of from 1 to 7 carbons, more preferably 1 to 4 carbons. The alkenyl group can be substituted or unsubstituted. When substituted the substituted group(s) is preferably, hydroxyl, cyano, alkoxy, ═O, ═S, NO₂, halogen, N(CH₃)₂, amino, or SH. The term “alkyl” also includes alkynyl groups that have an unsaturated hydrocarbon group containing at least one carbon-carbon triple bond, including straight-chain, branched-chain, and cyclic groups. Preferably, the alkynyl group has 1 to 12 carbons. More preferably, it is a lower alkynyl of from 1 to 7 carbons, more preferably 1 to 4 carbons. The alkynyl group can be substituted or unsubstituted. When substituted the substituted group(s) is preferably, hydroxyl, cyano, alkoxy, ═O, ═S, NO₂ or N(CH₃)₂, amino or SH.

Such alkyl groups can also include aryl, alkylaryl, carbocyclic aryl, heterocyclic aryl, amide and ester groups. An “aryl” group refers to an aromatic group that has at least one ring having a conjugated pi electron system and includes carbocyclic aryl, heterocyclic aryl and biaryl groups, all of which can be optionally substituted. The preferred substituent(s) of aryl groups are halogen, trihalomethyl, hydroxyl, SH, OH, cyano, alkoxy, alkyl, alkenyl, alkynyl, and amino groups. An “alkylaryl” group refers to an alkyl group (as described above) covalently joined to an aryl group (as described above). Carbocyclic aryl groups are groups wherein the ring atoms on the aromatic ring are all carbon atoms. The carbon atoms are optionally substituted. Heterocyclic aryl groups are groups having from 1 to 3 heteroatoms as ring atoms in the aromatic ring and the remainder of the ring atoms are carbon atoms. Suitable heteroatoms include oxygen, sulfinur, and nitrogen, and include furanyl, thienyl, pyridyl, pyrrolyl, N-lower alkyl pyrrolo, pyrimidyl, pyrazinyl, imidazolyl and the like, all optionally substituted. An “amide” refers to an —C(O)—NH—R, where R is either alkyl, aryl, alkylaryl or hydrogen. An “ester” refers to an —C(O)—OR′, where R is either alkyl, aryl, alkylaryl or hydrogen.

The term “nucleotide” as used herein is as recognized in the art to include natural bases (standard), and modified bases known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

In one embodiment, the invention features modified small-mer molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

By “abasic” is meant sugar moieties lacking a base or having other chemical groups in place of a base at the 1′ position, see for example Adamic et al., U.S. Pat. No. 5,998,203.

By “unmodified nucleoside” is meant one of the bases adenine, cytosine, guanine, thymine, uracil joined to the 1′ carbon of β-D-ribo-furanose.

By “modified nucleoside” is meant any nucleotide base which contains a modification in the chemical structure of an unmodified nucleotide base, sugar, phosphate or combination thereof.

In connection with 2′-modified nucleotides as described for the present invention, by “amino” is meant 2′-NH₂ or 2′-O—NH₂, which can be modified or unmodified. Such modified groups are described, for example, in Eckstein et al., U.S. Pat. No. 5,672,695 and Matulic-Adamic et al., U.S. Pat. No. 6,248,878, which are both incorporated by reference in their entireties.

Various modifications to nucleic acid small-mer structure can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

Administration of Nucleic Acid Molecules

A small-mer molecule of the invention can be adapted for use to treat viral infections such as HIV infection or diseases characterized by cellular proliferaction, such as cancer. For example, a small-mer molecule can comprise a delivery vehicle, including liposomes, for administration to a subject, carriers and diluents and their salts, and/or can be present in pharmaceutically acceptable formulations. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192, all of which are incorporated herein by reference. Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example Gonzalez et al., 1999, Bioconjugate Chem., 10, 1068-1074; Wang et al., International PCT publication Nos. WO 03/47518 and WO 03/46185), poly(lactic-co-glycolic)acid (PLGA) and PLCA microspheres (see for example U.S. Pat. No. 6,447,796 and US Patent Application Publication No. U.S. 2002130430), biodegradable nanocapsules, and bioadhesive microspheres, or by proteinaceous vectors (O'Hare and Normand, International PCT Publication No. WO 00/53722). In one embodiment, nucleic acid molecules or the invention are administered via biodegradable implant materials, such as elastic shape memory polymers (see for example Lendelein and Langer, 2002, Science, 296, 1673). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., 1999, Clin. Cancer Res., 5, 2330-2337 and Barry et al., International PCT Publication No. WO 99/31262. The molecules of the instant invention can be used as pharmaceutical agents. Pharmaceutical agents prevent, modulate the occurrence of, or treat (alleviate a symptom to some extent, preferably all of the symptoms) a disease state in a subject.

Thus, the invention features a pharmaceutical composition comprising one or more nucleic acid(s) of the invention in an acceptable carrier, such as a stabilizer, buffer, and the like. The small-mers of the invention can be administered (e.g., RNA, DNA or protein) and introduced into a subject by any standard means, with or without stabilizers, buffers, and the like, to form a pharmaceutical composition. When it is desired to use a liposome delivery mechanism, standard protocols for formation of liposomes can be followed. The compositions of the present invention can also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or subject, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity and forms that prevent the composition or formulation from exerting its effect.

By “systemic administration” is meant in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes expose the small-mer molecules of the invention to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation that can facilitate the association of drug with the surface of cells, such as, lymphocytes and macrophages is also useful. This approach can provide enhanced delivery of the drug to target cells by taking advantage of the specificity of macrophage and lymphocyte immune recognition of abnormal cells, such as cancer cells.

By “pharmaceutically acceptable formulation” is meant, a composition or formulation that allows for the effective distribution of the nucleic acid molecules of the instant invention in the physical location most suitable for their desired activity. Non-limiting examples of agents suitable for formulation with the nucleic acid molecules of the instant invention include: P-glycoprotein inhibitors (such as Pluronic P85), which can enhance entry of drugs into the CNS (Jolliet-Riant and Tillement, 1999, Fundam. Clin. Pharmacol., 13, 16-26); biodegradable polymers, such as poly (DL-lactide-coglycolide) microspheres for sustained release delivery after intracerebral implantation (Emerich, DF et al, 1999, Cell Transplant, 8, 47-58) (Alkermes, Inc. Cambridge, Mass.); and loaded nanoparticles, such as those made of polybutylcyanoacrylate, which can deliver drugs across the blood brain barrier and can alter neuronal uptake mechanisms (Prog Neuropsychopharmacol Biol Psychiatry, 23, 941-949, 1999). Other non-limiting examples of delivery strategies for the nucleic acid molecules of the instant invention include material described in Boado et al., 1998, J. Pharm. Sci., 87, 1308-1315; Tyler et al., 1999, FEBS Lett., 421, 280-284; Pardridge et al., 1995, PNAS USA., 92, 5592-5596; Boado, 1995, Adv. Drug Delivery Rev., 15, 73-107; Aldrian-Herrada et al., 1998, Nucleic Acids Res., 26, 4910-4916; and Tyler et al., 1999, PNAS USA., 96, 7053-7058.

The invention also features the use of the composition comprising surface-modified liposomes containing poly (ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwata et al., Chem. Pharm. Bull. 1995, 43, 1005-1011). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

The present invention also includes compositions prepared for storage or administration, which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A.R. Gennaro edit. 1985) hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

A pharmaceutically effective dose is that dose required to prevent, inhibit the occurrence of, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. Generally, an amount between about 0.1 mg/kg and about 100 mg/kg body weight/day of active ingredients is administered dependent upon potency of the negatively charged polymer.

The nucleic acid molecules of the invention and formulations thereof can be administered orally, topically, parenterally, by inhalation or spray, or rectally in dosage unit formulations containing conventional non-toxic pharmaceutically acceptable carriers, adjuvants and/or vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a nucleic acid molecule of the invention and a pharmaceutically acceptable carrier. One or more nucleic acid molecules of the invention can be present in association with one or more non-toxic pharmaceutically acceptable carriers and/or diluents and/or adjuvants, and if desired other active ingredients. The pharmaceutical compositions containing nucleic acid molecules of the invention can be in a form suitable for oral use, for example, as tablets, troches, lozenges, aqueous or oily suspensions, dispersible powders or granules, emulsion, hard or soft capsules, or syrups or elixirs.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example, gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example, polyoxyethylene sorbitan monooleate. The emulsions can also contain sweetening and flavoring agents.

Syrups and elixirs can be formulated with sweetening agents, for example, glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono-or diglycerides. In addition, fatty acids such as oleic acid find use in the preparation of injectables.

The nucleic acid molecules of the invention can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Nucleic acid molecules of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.1 mg to about 140 mg per kilogram of body weight per day are useful in the treatment of the above-indicated conditions (about 0.5 mg to about 7 g per subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The nucleic acid molecules of the present invention can also be administered to a subject in combination with other therapeutic compounds to increase the overall therapeutic effect. The use of multiple compounds to treat an indication can increase the beneficial effects while reducing the presence of side effects.

In one embodiment, the invention compositions are suitable for administering nucleic acid molecules of the invention to specific cell types. For example, the asialoglycoprotein receptor (ASGPr) (Wu and Wu, 1987, J. Biol. Chem. 262, 4429-4432) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, 1980, Cell, 22, 611-620; Connolly et al., 1982, J. Biol. Chem., 257, 939-945). Lee and Lee, 1987, Glycoconjugate J., 4, 317-328, obtained this high specificity through the use of N-acetyl-D-galactosamine as the carbohydrate moiety, which has higher affinity for the receptor, compared to galactose. This “clustering effect” has also been described for the binding and uptake of mannosyl-terminating glycoproteins or glycoconjugates (Ponpipom et al., 1981, J. Med. Chem., 24, 1388-1395). The use of galactose and galactosamine based conjugates to transport exogenous compounds across cell membranes can provide a targeted delivery approach to the treatment of liver disease such as HBV infection or hepatocellular carcinoma. The use of bioconjugates can also provide a reduction in the required dose of therapeutic compounds required for treatment. Furthermore, therapeutic bioavialability, pharmacodynamics, and pharmacokinetic parameters can be modulated through the use of nucleic acid bioconjugates of the invention. Non-limiting examples of such bioconjugates are described in Vargeese et al., U.S. Ser. No. 10/201,394, filed Aug. 13, 2001; and Matulic-Adamic et al., International PCT Publication No. WO 02/094185.

### EXAMPLES

The following are non-limiting examples showing the selection, isolation, synthesis and activity of nucleic acids of the instant invention.

### Example 1

### Design of Small-Mers

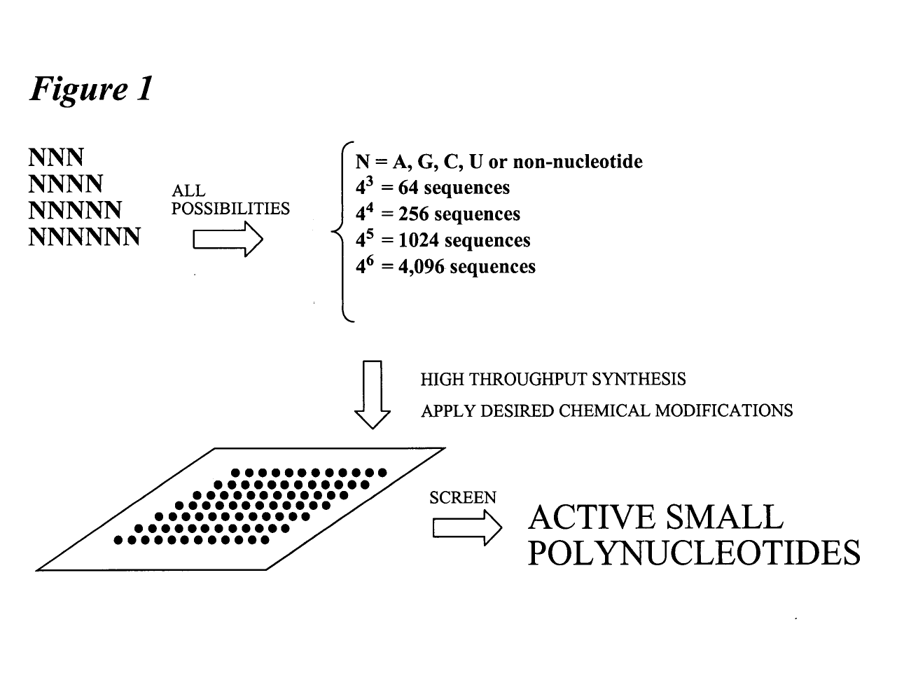

A high throughput combinatorial screening approach was used to develop small-mer having antiviral or antiproliferative activity. 2′-O-allyl modified nucleoside phosphoramidites were used to synthesize all possible sequence combinations (A, G, C, and U, see FIG. 1) of sequence space spanning 2 nucleotides to 5 nucleotides, all including phosphorothioate internucletide linkages and a 3′-terminal inverted deoxyabasic cap. Sequences were synthesized using solid phase oligonucleotide synthesis in a 96 well plate format as described herein. The resulting 1360 sequences were tested in a three cell line oncology screen provided by the National Cancer Institute (NCI) and a anti-HIV screen provided by (NCI). Methods suggested by NCI are described in Examples 4 and 5, however, these methods were modified such that the small-mers were resuspended in their growth medium and delivered at a final concentration of 5 uM for both the oncology and anti-HIV screenings.

### Example 2

### Oncology Screen

The following process in made avaiable by the Developmental Therapeutics Program NCI/NIH. The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 hours prior to addition of experimental drugs.

After 24 hours, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 hours at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM TRIZMA base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:
[(Ti−Tz)/(C−Tz)]×100 for concentrations for which Ti>/=Tz
[(Ti−Tz)/Tz]×100 for concentrations for which Ti<Tz.

Three dose response parameters are calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested. Sequences and data obtained from this screen are shown in Table III.

Three Cell Line Prescreen

In early 1995, during the course of reviewing data from the cancer screen, it became obvious that many agents were completely inactive under the conditions of the assay. A protocol for a 3 cell line prescreen was developed in collaboration with the Information Technology Branch (ITB, DTP). This prescreen tests for the presence of toxicity at 10⁻⁴M drug concentration and eliminates a large proportion of the inactive agents, but preserves “active” agents for multi-dose 60 cell line testing. Computer modeling indicated that approximately 50% of drugs could be eliminated by this prescreen without a significant decrease in ability to identify active agents, and should increase the throughput and efficiency of the main cancer screen with limited loss of information.

| Cell Line | Tumor Type |
|---|---|
| MCF7 | Breast |
| NCI-H460 | Lung |
| SF-268 | CNS |

### Example 3

### Oncology Screen Results

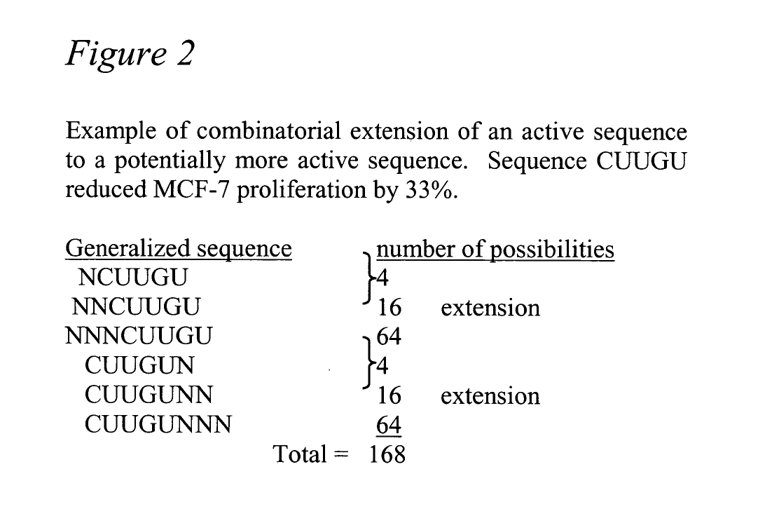

Of the fifteen 96-well plates of small-mers generated in Example 1, small-mers demonstrating 15% or more inhibition of proliferation in one of three cell types (MCF7, NCI-H460, or SF-268) are shown in Table III (92 oligos met this cutoff). These small-mers were resynthesized, and those molecules that repeated their level of anti proliferation activity were ranked in order of activity. The five most active were used as scaffold sequences for larger combinatorial libraries to improve antiproliverative activity. See FIG. 2 for an example sequence and the method of sequence extension.

An alternative approach is to extend the sequences that show activity to see if their effects are additive or synergistic. The order of sequences in a catanate can be tested in a combinatorial fashion and the method of linking the sequences can be optimized as well. Three possibilities are as follows: (1) direct linkage via phosphate or phosphorothioate bonds; (2)addition of unstructured sequence (any homopolymer of 2-20 bases) between active sequences; and (3) polyethylene glycol linkages of various lengths are placed between active sequences.

### Example 4

### Anti HIV Screen

The following process in made avaiable by the Developmental Therapeutics Program NCI/NIH. The anti-Human immunodeficieny virus (HIV) assay is a relatively simple method to determine the ability of a drug to protect cells against the cytopathic effects of HIV. T-lymphocyte-derived CEM cells are added to 96-well microtiter plates along with cell-free HIV and the test agent at ½-log dilutions over a multi-dose range. Six days after infection, a tetrazolium reagent, XTT, is added to the wells. In the presence of viable cells, XTT is metabolized to an orange colored formazan, such that the quantity of viable cells, and thus, the protective ability of the test agent, is proportional to the depth of the color. Uninfected cells are also treated with drug in order to determine the cytotoxicity of the drug, if any, to the CEM cells. The high throughput version of the assay was developed to allow the evaluation of chemical libraries. It is performed in 384-well plates at a single high dose, and does not include an addition of test agent to uninfected cells. Sequences and data obtained from this screen are shown in Table II.

### Example 5

### Anti-HIV Screen Results

The anti-HIV screen identified 88 sequences providing a >80% level of protection from HIV infection in CEM-ss cells, CD4+ T lymphocytes. A sequence alignment of these sequences indicates prominent homologies. Most notably, the family is defined by the presence of a GGG triplet. Dose responses are performed for ranking and revealing the most potent sequences. The five most active sequences are then tested in many different chemical variants; for example, all ribonucleotides, all deoxyribonucleotides, all 2′-Ome nucleotides, all 2′-F nucleotides, all 2′-NH2 nucleotides, +/− phosphorothioate linkages, dithioate linkages, or phosphonate linkages. In addition, the same combinatorial approach to improving potency as outlined above for the anti-cancer sequences can be applied to the anti-HIV sequences.

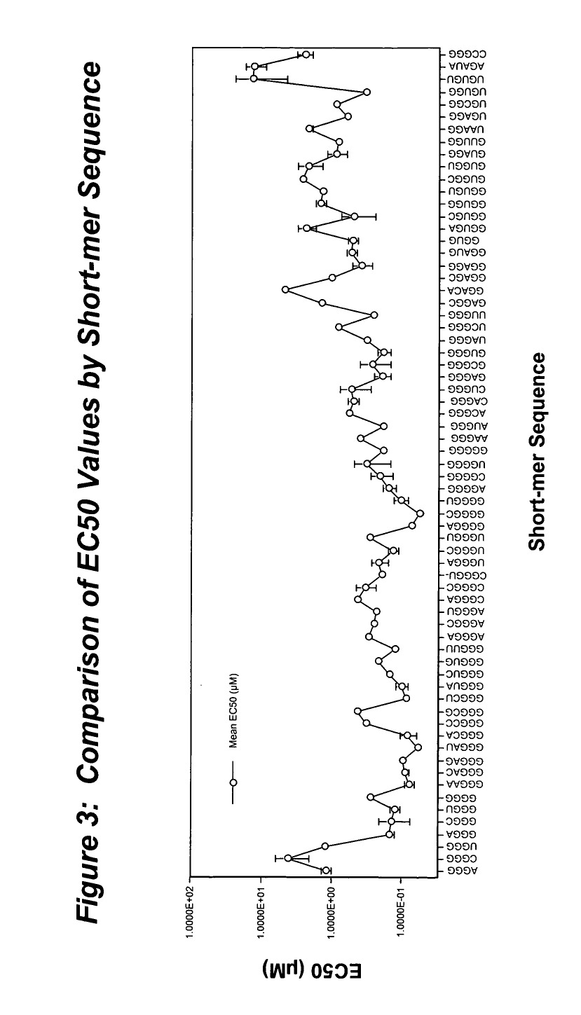

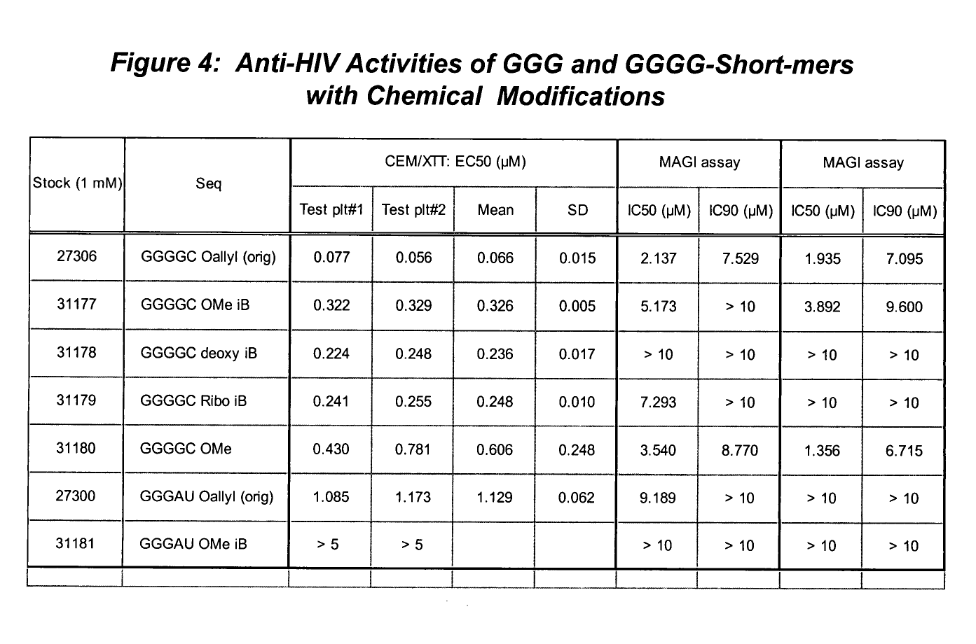

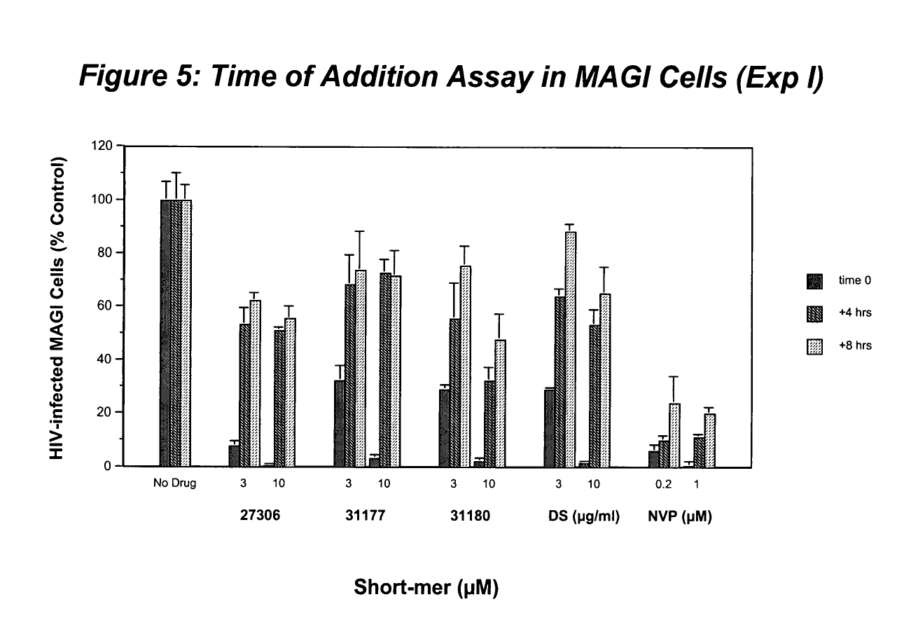

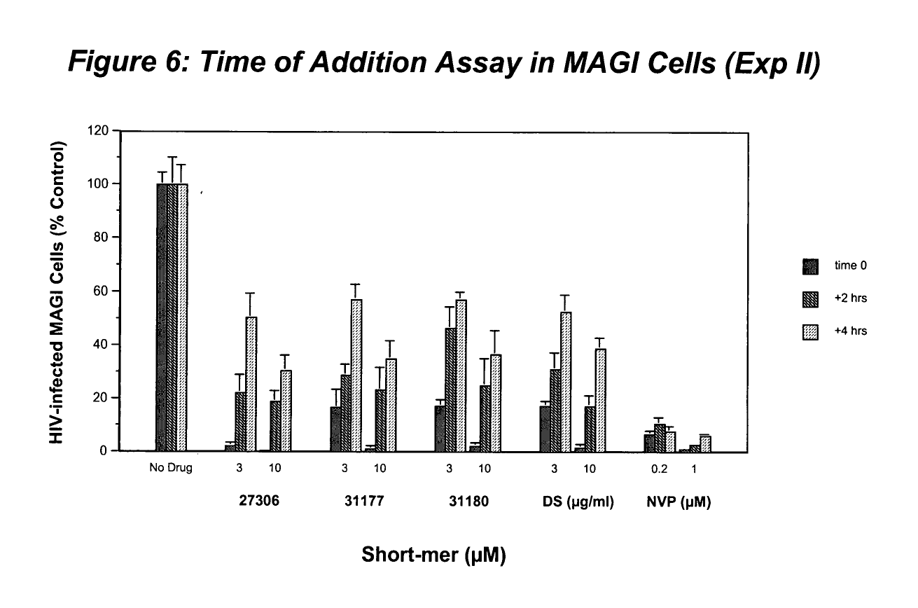

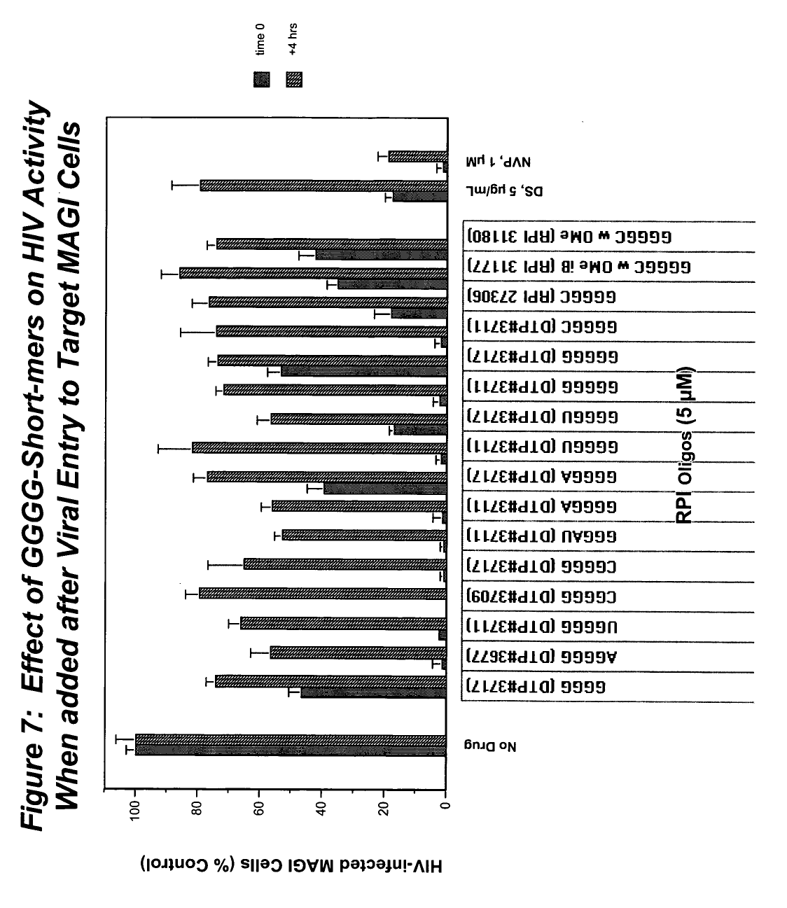

Smallmer sequences were also tested for anti-HIV activity in a multinuclear activation of galactosidase-indicator gene (MAGI) assay. MAGI cells are CD4+ HeLa cells that have been engineered to express beta gal gene via Tat-mediated LTR transactivation. The number of HIV infected cells is counted in the assay, which was optimized to examine just one round of HIV infection. As such, the assay involves one round of HIV infection from viral entry to viral integration. No multiplicity of infection results because the assay is stopped before the second round takes place. EC50 values were determined based on smallmer sequence (see FIG. 3, sequences are shown without the terminal 3′-deoxyabasic moiety). IC50 values in the MAGI assay tend to be higher than in the CEM based assay, because higher viral inoculum is used to get an acceptable endpoint. The activities of various smallmers with differing chemical modifications was determined (see FIG. 4). After confirming the activity of GGGGC smallmers (47082, 47083, and 47086 all shown without terminal 3′-deoxyabasic moiety), these smallmers were tested in a time of addition assay in MAGI cells (see FIGS. 5 and 6). In a repeated experiment it was observed that the smallmers drastically lost antiviral activity when the addition was delayed for 2 to 4 hours, suggesting that the smallmers are blocking viral entry (see FIG. 7). In FIG. 7, DS stands for dextran sulfate, a reference drug for viral entry blockade and NVP stands for nevirapine, a reverse transcriptase inhibitor.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications can be made to the invention disclosed herein without departing from the scope and spirit of the invention. Thus, such additional embodiments are within the scope of the present invention and the following claims.

The invention illustratively described herein suitably can be practiced in the absence of any element or elements, limitation or limitations that are not specifically disclosed herein. Thus, for example, in each instance herein any of the terms “comprising”, “consisting essentially of” and “consisting of” can be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed can be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

TABLE I
small-mer sequences

| | | |
|---|---|---|
| RPI# | Sequence | SeqID |

| | | |
|---|---|---|
| 26289 | aₛaₛ B | 1 |
| 26295 | cₛgₛ B | 2 |
| 26303 | uₛgₛ B | 3 |
| 26304 | uₛuₛ B | 4 |
| 26305 | aₛaₛaₛ B | 5 |
| 26347 | gₛgₛgₛ B | 6 |
| 26362 | uₛgₛcₛ B | 7 |
| 26366 | uₛuₛcₛ B | 8 |
| 26368 | uₛuₛuₛ B | 9 |
| 26369 | cₛgₛaₛaₛ B | 10 |
| 26379 | cₛgₛgₛgₛ B | 11 |
| 26441 | gₛgₛgₛaₛ B | 12 |
| 26442 | gₛgₛgₛcₛ B | 13 |
| 26443 | gₛgₛgₛgₛ B | 14 |
| 26444 | gₛgₛgₛuₛ B | 15 |
| 26447 | gₛgₛuₛgₛ B | 16 |
| 26456 | gₛuₛcₛuₛ B | 17 |
| 26462 | gₛuₛuₛcₛ B | 18 |
| 26465 | aₛaₛaₛaₛ B | 19 |
| 26467 | aₛaₛaₛgₛ B | 20 |
| 26469 | aₛaₛcₛaₛ B | 21 |
| 26471 | aₛaₛcₛgₛ B | 22 |
| 26473 | aₛaₛgₛaₛ B | 23 |
| 26475 | aₛaₛgₛgₛ B | 24 |
| 26477 | aₛaₛuₛaₛ B | 25 |
| 26501 | aₛgₛcₛaₛ B | 26 |
| 26502 | aₛgₛcₛcₛ B | 27 |
| 26507 | aₛgₛgₛgₛ B | 28 |
| 26525 | aₛuₛuₛaₛ B | 29 |
| 26537 | cₛaₛgₛaₛ B | 30 |
| 26549 | cₛcₛcₛaₛ B | 31 |
| 26550 | cₛcₛcₛcₛ B | 32 |
| 26552 | cₛcₛcₛuₛ B | 33 |
| 26554 | cₛcₛgₛcₛ B | 34 |
| 26558 | cₛcₛuₛcₛ B | 35 |
| 26560 | cₛcₛuₛuₛ B | 36 |
| 26561 | uₛaₛaₛaₛ B | 37 |
| 26576 | uₛaₛuₛuₛ B | 38 |
| 26603 | uₛgₛgₛgₛ B | 39 |
| 26630 | aₛaₛaₛcₛcₛ B | 40 |
| 26667 | aₛaₛgₛgₛgₛ B | 41 |
| 26729 | aₛcₛgₛgₛaₛ B | 42 |
| 26731 | aₛcₛgₛgₛgₛ B | 43 |
| 26742 | aₛcₛuₛcₛcₛ B | 44 |
| 26753 | aₛgₛaₛaₛaₛ B | 45 |
| 26755 | aₛgₛaₛaₛgₛ B | 46 |
| 26757 | aₛgₛaₛcₛaₛ B | 47 |
| 26759 | aₛgₛaₛcₛgₛ B | 48 |
| 26763 | aₛgₛaₛgₛgₛ B | 49 |
| 26765 | aₛgₛaₛuₛaₛ B | 50 |
| 26768 | aₛgₛaₛuₛuₛ B | 51 |
| 26772 | aₛgₛcₛaₛuₛ B | 52 |
| 26775 | aₛgₛcₛcₛgₛ B | 53 |
| 26778 | aₛgₛcₛgₛcₛ B | 54 |
| 26779 | aₛgₛcₛgₛgₛ B | 55 |
| 26788 | aₛgₛgₛaₛuₛ B | 56 |
| 26793 | aₛgₛgₛgₛaₛ B | 57 |
| 26794 | aₛgₛgₛgₛcₛ B | 58 |
| 26795 | aₛgₛgₛgₛgₛ B | 59 |
| 26796 | aₛgₛgₛgₛuₛ B | 60 |
| 26797 | aₛgₛgₛuₛaₛ B | 61 |
| 26825 | aₛuₛaₛgₛaₛ B | 62 |
| 26838 | aₛuₛcₛcₛcₛ B | 63 |
| 26840 | aₛuₛcₛcₛuₛ B | 64 |
| 26842 | aₛuₛcₛgₛcₛ B | 65 |
| 26846 | aₛuₛcₛuₛcₛ B | 66 |
| 26848 | aₛuₛcₛuₛuₛ B | 67 |
| 26857 | aₛuₛgₛgₛaₛ B | 68 |
| 26859 | aₛuₛgₛgₛgₛ B | 69 |
| 26875 | aₛuₛuₛgₛgₛ B | 70 |
| 26876 | aₛuₛuₛgₛuₛ B | 71 |
| 26881 | cₛaₛaₛaₛaₛ B | 72 |
| 26892 | cₛaₛaₛgₛuₛ B | 73 |
| 26921 | cₛaₛgₛgₛaₛ B | 74 |
| 26923 | cₛaₛgₛgₛgₛ B | 74 |
| 26934 | cₛaₛuₛcₛcₛ B | 76 |
| 26939 | cₛaₛuₛgₛgₛ B | 77 |
| 26940 | cₛaₛuₛgₛuₛ B | 78 |
| 26945 | cₛcₛaₛaₛaₛ B | 79 |
| 26946 | cₛcₛaₛaₛcₛ B | 80 |
| 26947 | cₛcₛaₛgₛgₛ B | 81 |
| 26953 | cₛcₛaₛgₛaₛ B | 82 |
| 26955 | cₛcₛaₛgₛgₛ B | 83 |
| 26970 | cₛcₛcₛgₛcₛ B | 84 |
| 26987 | cₛcₛgₛgₛcₛ B | 85 |
| 26993 | cₛcₛuₛaₛaₛ B | 86 |
| 27025 | cₛgₛcₛaₛaₛ B | 87 |
| 27029 | cₛgₛcₛcₛaₛ B | 88 |
| 27030 | cₛgₛcₛcₛcₛ B | 89 |
| 27032 | cₛgₛcₛcₛuₛ B | 90 |
| 27034 | cₛgₛcₛgₛcₛ B | 91 |
| 27040 | cₛgₛcₛuₛuₛ B | 92 |
| 27041 | cₛgₛgₛaₛaₛ B | 93 |
| 27042 | cₛgₛgₛaₛcₛ B | 94 |
| 27043 | cₛgₛgₛaₛgₛ B | 95 |
| 27045 | cₛgₛgₛcₛaₛ B | 96 |
| 27049 | cₛgₛgₛgₛaₛ B | 97 |
| 27050 | cₛgₛgₛgₛcₛ B | 98 |
| 27051 | cₛgₛgₛgₛgₛ B | gg |
| 27052 | cₛgₛgₛgₛuₛ B | 100 |
| 27066 | cₛgₛuₛgₛcₛ B | 101 |
| 27070 | cₛgₛuₛuₛcₛ B | 102 |
| 27089 | cₛuₛcₛaₛaₛ B | 103 |
| 27090 | cₛuₛcₛaₛcₛ B | 104 |
| 27102 | cₛuₛcₛuₛcₛ B | 105 |
| 27103 | cₛuₛcₛuₛgₛ B | 106 |
| 27104 | cₛuₛcₛuₛuₛ B | 107 |
| 27114 | cₛuₛgₛgₛcₛ B | 108 |
| 27115 | cₛuₛgₛgₛgₛ B | 109 |
| 27125 | cₛuₛuₛcₛaₛ B | 110 |
| 27126 | cₛuₛuₛcₛcₛ B | 111 |
| 27128 | cₛuₛuₛcₛuₛ B | 112 |
| 27130 | cₛuₛuₛgₛcₛ B | 113 |
| 27132 | cₛuₛuₛgₛuₛ B | 114 |
| 27134 | cₛuₛuₛuₛcₛ B | 115 |
| 27136 | cₛuₛuₛuₛuₛ B | 116 |
| 27177 | gₛaₛgₛgₛaₛ B | 117 |
| 27178 | gₛaₛgₛgₛcₛ B | 118 |
| 27179 | gₛaₛgₛgₛgₛ B | 119 |
| 27243 | gₛcₛgₛgₛgₛ B | 120 |
| 27269 | gₛgₛaₛcₛaₛ B | 121 |
| 27274 | gₛgₛaₛgₛcₛ B | 122 |
| 27275 | gₛgₛaₛgₛgₛ B | 123 |
| 27279 | gₛgₛaₛuₛgₛ B | 124 |
| 27295 | gₛgₛcₛuₛgₛ B | 125 |
| 27297 | gₛgₛgₛaₛaₛ B | 126 |
| 27298 | gₛgₛgₛaₛcₛ B | 127 |
| 27299 | gₛgₛgₛaₛgₛ B | 128 |
| 27300 | gₛgₛgₛaₛuₛ B | 129 |
| 27301 | gₛgₛgₛcₛaₛ B | 130 |
| 27302 | gₛgₛgₛcₛcₛ B | 131 |
| 27303 | gₛgₛgₛcₛgₛ B | 132 |
| 27304 | gₛgₛgₛcₛuₛ B | 133 |
| 27305 | gₛgₛgₛgₛaₛ B | 134 |
| 27306 | gₛgₛgₛgₛcₛ B | 135 |
| 27307 | gₛgₛgₛgₛgₛ B | 136 |
| 27308 | gₛgₛgₛgₛuₛ B | 137 |
| 27309 | gₛgₛgₛuₛaₛ B | 138 |
| 27310 | gₛgₛgₛuₛcₛ B | 139 |
| 27311 | gₛgₛgₛuₛgₛ B | 140 |
| 27312 | gₛgₛgₛuₛuₛ B | 141 |
| 27321 | gₛgₛuₛgₛaₛ B | 142 |
| 27322 | gₛgₛuₛgₛcₛ B | 143 |
| 27323 | gₛgₛuₛgₛgₛ B | 144 |
| 27324 | gₛgₛuₛgₛuₛ B | 145 |
| 27339 | gₛuₛaₛgₛgₛ B | 146 |
| 27340 | gₛuₛaₛgₛuₛ B | 147 |
| 27352 | gₛuₛcₛcₛuₛ B | 148 |
| 27370 | gₛuₛgₛgₛcₛ B | 149 |
| 27371 | gₛuₛgₛgₛgₛ B | 150 |
| 27372 | gₛuₛgₛgₛuₛ B | 151 |
| 27376 | gₛuₛgₛuₛuₛ B | 152 |
| 27387 | gₛuₛuₛgₛgₛ B | 153 |
| 27388 | gₛuₛuₛgₛuₛ B | 154 |
| 27395 | uₛaₛaₛaₛgₛ B | 155 |
| 27403 | uₛaₛaₛgₛgₛ B | 156 |
| 27412 | uₛaₛcₛaₛuₛ B | 157 |
| 27424 | uₛaₛcₛuₛuₛ B | 158 |
| 27435 | uₛaₛgₛgₛgₛ B | 159 |
| 27462 | uₛcₛaₛcₛcₛ B | 160 |
| 27474 | uₛcₛcₛaₛcₛ B | 161 |
| 27485 | uₛcₛcₛuₛaₛ B | 162 |
| 27486 | uₛcₛcₛuₛcₛ B | 163 |
| 27497 | uₛcₛgₛgₛaₛ B | 164 |
| 27499 | uₛcₛgₛgₛgₛ B | 165 |
| 27510 | uₛcₛuₛcₛcₛ B | 166 |
| 27512 | uₛcₛuₛcₛuₛ B | 167 |
| 27516 | uₛcₛuₛgₛuₛ B | 168 |
| 27520 | uₛcₛuₛuₛuₛ B | 169 |
| 27531 | uₛgₛaₛgₛgₛ B | 170 |
| 27547 | uₛgₛcₛgₛgₛ B | 171 |
| 27561 | uₛgₛgₛgₛaₛ B | 172 |
| 27562 | uₛgₛgₛgₛcₛ B | 173 |
| 27563 | uₛgₛgₛgₛgₛ B | 174 |
| 27564 | uₛgₛgₛgₛuₛ B | 175 |
| 27574 | uₛgₛuₛcₛcₛ B | 176 |
| 27579 | uₛgₛuₛgₛgₛ B | 177 |
| 27580 | uₛgₛuₛgₛuₛ B | 178 |
| 27594 | uₛuₛaₛgₛcₛ B | 179 |
| 27608 | uₛuₛcₛcₛuₛ B | 180 |
| 27611 | uₛuₛcₛgₛgₛ B | 181 |
| 27627 | uₛuₛgₛgₛgₛ B | 182 |

a = 2′-O-allyl adenosine
g = 2′-O-allyl guanosine
u = 2′-O-allyl uridine
c = 2′-O-allyl cytidine
B = inverted deoxyabasic
S =phosphorothioate inter-nucleotide linkage

TABLE II
small-mer anti-HIV sequences

| | | | | | | |
|---|---|---|---|---|---|---|
| | | Plate | % pro- | Plate | % pro- | |
| Sequence | RPI# | # pos | tection | # pos | tection | Seq ID |

| | | | | | | |
|---|---|---|---|---|---|---|
| aₛgₛgₛgₛ B | 26507 | 1222 D7 | 52 | 1271 G1 | 80 | 28 |
| cₛgₛgₛgₛ B | 26379 | 1221 A11 | 59 | 1271 A11 | 107 | 11 |
| uₛgₛgₛgₛ B | 26603 | 1223 F3 | 95 | 1271 H1 | 93 | 39 |
| gₛgₛgₛ B | 26347 | 1220 F5 | 105 | | | 6 |
| | | 1234 H1 | 101 | | | |
| gₛgₛgₛ B | 26347 | 6 | | | | |
| gₛgₛgₛaₛ B | 26441 | 1221 G1 | 96 | 1269 B1 | 106 | 12 |
| | | 1234 E5 | 111 | | | |
| gₛgₛgₛcₛ B | 26442 | 1221 G2 | 99 | 1271 C1 | 91 | 13 |
| | | 1234 E6 | 102 | | | |
| gₛgₛgₛuₛ B | 26444 | 1221 G4 | 99 | 1271 E1 | 98 | 15 |
| | | 1234 F1 | 108 | | | |
| gₛgₛgₛaₛ B | 26441 | 1221 G1 | 96 | 1271 B1 | 106 | 12 |
| | | 1234 E5 | 111 | | | |
| gₛgₛgₛcₛ B | 26442 | 1221 G2 | 99 | 1271 C1 | 91 | 13 |
| | | 1234 E6 | 101 | | | |
| gₛgₛgₛgₛ B | 26443 | 1221 G3 | 100 | 1271 D1 | 104 | 14 |
| gₛgₛgₛuₛ B | 26444 | 1221 G4 | 99 | 1271 E1 | 97 | 15 |
| | | 1234 F1 | 109 | | | |
| gₛgₛgₛaₛaₛ B | 27297 | 1230 F5 | 106 | 1271 D8 | 105 | 126 |
| gₛgₛgₛaₛcₛ B | 27298 | 1230 F6 | 104 | 1271 D9 | 91 | 127 |
| gₛgₛgₛaₛgₛ B | 27299 | 1230 F7 | 111 | 1271 D10 | 94 | 128 |
| gₛgₛgₛaₛuₛ B | 27300 | 1230 F8 | 101 | 1271 D11 | 98 | 129 |
| gₛgₛgₛcₛaₛ B | 27301 | 1230 F9 | 114 | 1271 E2 | 98 | 130 |
| gₛgₛgₛcₛcₛ B | 27302 | 1230 F10 | 101 | 1271 E3 | 96 | 131 |
| gₛgₛgₛcₛgₛ B | 27303 | 1230 F11 | 111 | 1271 E4 | 98 | 132 |
| gₛgₛgₛcₛuₛ B | 27304 | 1230 F12 | 116 | 1271 E5 | 96 | 133 |
| gₛgₛgₛuₛaₛ B | 27309 | 1230 G5 | 98 | 1271 E10 | 97 | 138 |
| gₛgₛgₛuₛcₛ B | 27310 | 1230 G6 | 99 | 1271 E11 | 95 | 139 |
| gₛgₛgₛuₛgₛ B | 27311 | 1230 G7 | 98 | 1271 F2 | 103 | 140 |
| gₛgₛgₛuₛuₛ B | 27312 | 1230 G8 | 100 | 1271 F3 | 96 | 141 |
| aₛgₛgₛgₛaₛ B | 26793 | 1225 D5 | 92 | 1271 A10 | 102 | 57 |
| aₛgₛgₛgₛcₛ B | 26794 | 1225 D6 | 99 | 1271 A11 | 109 | 58 |
| aₛgₛgₛgₛuₛ B | 26796 | 1225 D8 | 93 | 1271 B3 | 113 | 60 |
| cₛgₛgₛgₛaₛ B | 27049 | 1228 A9 | 100 | 1271 C6 | 96 | 97 |
| | | | | 1272 B12 | | |
| cₛgₛgₛgₛcₛ B | 27050 | 1228 A10 | 100 | 1271 C7 | 91 | 98 |
| cₛgₛgₛgₛuₛ B | 27052 | 1228 A12 | 99 | 1271 C9 | 88 | 100 |
| uₛgₛgₛgₛaₛ B | 27561 | 1233 D5 | 95 | 1271 H3 | 83 | 172 |
| uₛgₛgₛgₛcₛ B | 27562 | 1233 D6 | 92 | 1271 H4 | 87 | 173 |
| uₛgₛgₛgₛuₛ B | 27564 | 1233 D8 | 101 | 1271 H6 | 85 | 175 |
| gₛgₛgₛgₛaₛ B | 27305 | 1230 G1 | 91 | 1271 E6 | 94 | 134 |
| gₛgₛgₛgₛcₛ B | 27306 | 1230 G2 | 81 | 1271 E7 | 95 | 135 |
| gₛgₛgₛgₛuₛ B | 27308 | 1230 G4 | 99 | 1271 E9 | 89 | 137 |
| | | | | 1272 D11 | | |
| aₛgₛgₛgₛgₛ B | 26795 | 1225 D7 | 92 | 1271 B2 | 110 | 59 |
| cₛgₛgₛgₛgₛ B | 27051 | 1228 A11 | 96 | 1271 C8 | 91 | 99 |
| | | | | 1272 C4 | | |
| uₛgₛgₛgₛgₛ B | 27563 | 1233 D7 | 93 | 1271 H5 | 87 | 174 |
| gₛgₛgₛgₛgₛ B | 27307 | 1230 G3 | 86 | 1271 E8 | 99 | 136 |
| aₛaₛgₛgₛgₛ B | 26667 | 1224 A11 | 97 | 1271 A2 | 106 | 41 |
| aₛuₛgₛgₛgₛ B | 26859 | 1226 A11 | 92 | 1271 B7 | 99 | 69 |
| aₛcₛgₛgₛgₛ B | 26731 | 1224 G3 | 105 | 1271 A3 | 114 | 43 |
| cₛaₛgₛgₛgₛ B | 26923 | 1226 G3 | 105 | 1271 C3 | 94 | 75 |
| cₛuₛgₛgₛgₛ B | 27115 | 1228 G3 | 107 | 1271 C10 | 89 | 109 |
| gₛaₛgₛgₛgₛ B | 27179 | 1229 D7 | 94 | 1271 D2 | 98 | 119 |
| gₛcₛgₛgₛgₛ B | 27243 | 1230 A11 | 110 | 1271 D3 | 100 | 120 |
| gₛuₛgₛgₛgₛ B | 27371 | 1231 D7 | 88 | 1271 G2 | 78 | 150 |
| uₛaₛgₛgₛgₛ B | 27435 | 1232 A11 | 95 | 1271 G10 | 84 | 159 |
| uₛcₛgₛgₛgₛ B | 27499 | 1232 G3 | 110 | 1271 H11 | 86 | 165 |
| uₛuₛgₛgₛgₛ B | 27627 | 1234 D11 | 104 | 1271 H10 | 80 | 182 |
| gₛaₛgₛgₛcₛ B | 27178 | 1229 D6 | 91 | 1271 C11 | 97 | 118 |
| gₛgₛaₛcₛaₛ B | 27269 | 1230 D1 | 94 | 1271 D4 | 101 | 121 |
| gₛgₛaₛgₛcₛ B | 27274 | 1230 D6 | 102 | 1271 D5 | 106 | 122 |
| gₛgₛaₛgₛgₛ B | 27275 | 1230 D7 | 101 | 1271 D6 | 102 | 123 |
| | | | | 1273 A1..F1 | | |
| gₛgₛaₛuₛgₛ B | 27279 | 1230 D11 | 111 | 1271 D7 | 113 | 124 |
| | | | | 1272 D9 | | |
| gₛgₛuₛgₛ B | 26447 | 1221 G7 | 111 | 1271 F1 | 99 | 16 |
| | | 1234 F2 | 89 | | | |
| gₛgₛuₛgₛ B | 26447 | | | 1271 | | 16 |
| gₛgₛuₛgₛaₛ B | 27321 | 1230 H5 | 92 | 1271 F4 | 94 | 142 |
| gₛgₛuₛgₛcₛ B | 27322 | 1230 H6 | 93 | 1271 F5 | 99 | 143 |
| gₛgₛuₛgₛgₛ B | 27323 | 1230 H7 | 95 | 1271 F6 | 90 | 144 |
| gₛgₛuₛgₛuₛ B | 27324 | 1230 H8 | 97 | 1271 F7 | 92 | 145 |
| | | | | 1272 E4 | | |
| gₛuₛgₛgₛcₛ B | 27370 | 1231 D6 | 89 | 1271 F11 | 77 | 149 |
| gₛuₛgₛgₛuₛ B | 27372 | 1231 D8 | 92 | 1271 G3 | 83 | 151 |
| gₛuₛgₛuₛuₛ B | 27376 | 1231 D12 | 99 | 1271 G4 | 50 | 152 |
| gₛuₛaₛgₛgₛ B | 27339 | 1231 A11 | 121 | 1271 F8 | 85 | 146 |
| gₛuₛaₛgₛuₛ B | 27340 | 1231 A12 | 128 | 1271 F9 | 26 | 147 |
| gₛuₛcₛcₛuₛ B | 27352 | 1231 B12 | 89 | 1271 F10 | 12 | 148 |
| gₛuₛuₛgₛgₛ B | 27387 | 1231 E11 | 103 | 1271 G5 | 94 | 153 |
| gₛuₛuₛgₛuₛ B | 27388 | 1231 E12 | 106 | 1271 G6 | 14 | 154 |
| uₛaₛaₛgₛgₛ B | 27403 | 1231 G3 | 105 | 1271 G7 | 97 | 156 |
| uₛaₛcₛaₛuₛ B | 27412 | 1231 G12 | 109 | 1271 G8 | 20 | 157 |
| uₛaₛcₛuₛuₛ B | 27424 | 1231 H12 | 101 | 1271 G9 | 22 | 158 |
| uₛgₛaₛgₛgₛ B | 27531 | 1233 A11 | 109 | 1271 G11 | 86 | 170 |
| uₛgₛcₛgₛgₛ B | 27547 | 1233 C3 | 106 | 1271 H2 | 96 | 171 |
| uₛgₛuₛcₛcₛ B | 27574 | 1233 E6 | 83 | 1271 H7 | 22 | 176 |
| uₛgₛuₛgₛgₛ B | 27579 | 1233 E11 | 103 | 1271 H8 | 85 | 177 |
| | | | | 1272 F4 | | |
| uₛgₛuₛgₛuₛ B | 27580 | 1233 E12 | 86 | 1271 H9 | 59 | 178 |
| cₛaₛuₛgₛgₛ B | 26939 | 1226 H7 | 107 | 1271 C4 | 48 | 77 |
| cₛaₛuₛgₛuₛ B | 26940 | 1226 H8 | 86 | 1271 C5 | 22 | 78 |
| cₛaₛgₛgₛaₛ B | 26921 | 1226 G1 | 112 | 1271 C2 | 38 | 74 |
| cₛaₛaₛgₛuₛ B | 26892 | 1226 D8 | 86 | 1271 B11 | −2 | 73 |
| cₛaₛaₛaₛaₛ B | 26881 | 1226 C9 | 88 | 1271 B10 | 4 | 72 |
| aₛuₛuₛgₛgₛ B | 26875 | 1226 C3 | 89 | 1271 B8 | 51 | 70 |
| aₛuₛuₛgₛuₛ B | 26876 | 1226 C4 | 81 | 1271 B9 | 35 | 71 |
| aₛuₛgₛgₛaₛ B | 26857 | 1226 A9 | 97 | 1271 B6 | 26 | 68 |
| aₛuₛaₛgₛaₛ B | 26825 | 1225 G1 | 103 | 1271 B5 | 11 | 62 |
| aₛgₛaₛuₛaₛ B | 26765 | 1225 B1 | 113 | 1271 A4 | 61 | 50 |
| aₛgₛaₛuₛuₛ B | 26768 | 1225 B4 | 113 | 1271 A5 | 51 | 51 |
| aₛgₛcₛaₛuₛ B | 26772 | 1225 B8 | 88 | 1271 A6 | 48 | 52 |
| aₛgₛcₛcₛgₛ B | 26775 | 1225 B11 | 98 | 1271 A7 | 24 | 53 |
| aₛgₛcₛgₛgₛ B | 26779 | 1225 C3 | 92 | 1271 A8 | 83 | 55 |
| aₛgₛgₛaₛuₛ B | 26788 | 1225 C12 | 82 | 1271 A9 | 63 | 56 |
| aₛgₛgₛuₛaₛ B | 26797 | 1225 D9 | 80 | 1271 B4 | 30 | 61 |
| uₛaₛaₛaₛgₛ B | 27395 | 1231 F7 | 10 | 1271 A12 | 14 | 155 |
| | | | | 1271 G12 | 18 | |
| uₛuₛaₛgₛcₛ B | 27594 | 1233 G2 | 0 | 1271 B12 | 13 | 179 |
| | | | | 1271 H12 | 17 | |
| cₛuₛcₛuₛuₛ B | 27104 | 1228 F4 | 0 | 1271 C12 | 17 | 107 |
| cₛcₛgₛgₛgₛ B | 26987 | 1227 D7 | 69 | 1271 D12 | 84 | 85 |
| cₛgₛuₛuₛcₛ B | 27070 | 1228 6 | | 1271 E12 | 13 | 102 |
| uₛcₛuₛcₛcₛ B | 27510 | 1232 H2 | 0 | 1271 F12 | 18 | 166 |

a = 2′-O-allyl adenosine
g = 2′-O-allyl guanosine
u = 2′-O-allyl uridine
c = 2′-O-allyl cytidine
B = inverted deoxyabasic
S = phosphorothioate internucleotide linkage

TABLE III
small-mer anti-oncology sequences

| | | | | | | | |
|---|---|---|---|---|---|---|---|
| | | | | | NCI- | | Seq |
| Sequence | RPI# | Plate | Well | MCF7 | H460 | SF-268 | ID |

| | | | | | | | |
|---|---|---|---|---|---|---|---|
| aₛaₛ B | 26289 | 1220 | A01 | 8 | 89 | 97 | 1 |
| aₛaₛaₛ B | 26305 | 1220 | A03 | 84 | 90 | 100 | 5 |
| cₛgₛ B | 26295 | 1220 | D01 | 83 | 105 | 141 | 2 |
| uₛgₛ B | 26303 | 1220 | H01 | 78 | 106 | 99 | 3 |
| uₛuₛ B | 26304 | 1220 | H02 | 1 | 94 | 102 | 4 |
| uₛgₛcₛ B | 26362 | 1220 | H04 | 80 | 95 | 102 | 7 |
| uₛuₛcₛ B | 26366 | 1220 | H08 | 81 | 96 | 102 | 8 |
| uₛuₛuₛ B | 26368 | 1220 | H10 | 78 | 90 | 95 | 9 |
| aₛaₛaₛaₛ B | 26465 | 1222 | A01 | 17 | 93 | 100 | 19 |
| aₛaₛaₛgₛ B | 26467 | 1222 | A03 | 72 | 85 | 101 | 20 |
| aₛaₛcₛaₛ B | 26469 | 1222 | A05 | 83 | 95 | 95 | 21 |
| aₛaₛcₛgₛ B | 26471 | 1222 | A07 | 74 | 81 | 107 | 22 |
| aₛaₛgₛaₛ B | 26473 | 1222 | A09 | 96 | 78 | 85 | 23 |
| aₛaₛgₛgₛ B | 26475 | 1222 | A11 | 74 | 97 | 102 | 24 |
| aₛaₛuₛaₛ B | 26477 | 1222 | B01 | 81 | 109 | 122 | 25 |
| aₛgₛcₛaₛ B | 26501 | 1222 | D01 | 70 | 107 | 133 | 26 |
| aₛgₛcₛcₛ B | 26502 | 1222 | D02 | 78 | 102 | 137 | 27 |
| aₛuₛuₛaₛ B | 26525 | 1222 | F01 | 75 | 104 | 145 | 29 |
| cₛaₛgₛaₛ B | 26537 | 1222 | G01 | 82 | 106 | 121 | 30 |
| cₛcₛcₛaₛ B | 26549 | 1222 | H01 | 75 | 107 | 102 | 31 |
| cₛcₛcₛcₛ B | 26550 | 1222 | H02 | 0 | 93 | 111 | 32 |
| cₛcₛcₛuₛ B | 26552 | 1222 | H04 | 73 | 94 | 113 | 33 |
| cₛcₛgₛcₛ B | 26554 | 1222 | H06 | 81 | 90 | 108 | 34 |
| cₛcₛuₛcₛ B | 26558 | 1222 | H10 | 78 | 90 | 106 | 35 |
| cₛcₛuₛuₛ B | 26560 | 1222 | H12 | 74 | 97 | 107 | 36 |
| cₛcₛaₛaₛaₛ B | 26945 | 1227 | A01 | 1 | 85 | 98 | 79 |
| cₛcₛaₛaₛcₛ B | 26946 | 1227 | A02 | 82 | 105 | 107 | 80 |
| cₛcₛaₛaₛgₛ B | 26947 | 1227 | A03 | 78 | 87 | 97 | 81 |
| cₛcₛaₛgₛaₛ B | 26953 | 1227 | A09 | 79 | 92 | 91 | 82 |
| cₛcₛaₛgₛgₛ B | 26955 | 1227 | A11 | 85 | 91 | 101 | 83 |
| cₛcₛcₛgₛcₛ B | 26970 | 1227 | C02 | 82 | 103 | 127 | 84 |
| cₛcₛuₛaₛaₛ B | 26993 | 1227 | E01 | 82 | 99 | 138 | 86 |
| cₛgₛcₛaₛaₛ B | 27025 | 1227 | G09 | 84 | 105 | 121 | 87 |
| cₛgₛcₛcₛaₛ B | 27029 | 1227 | H01 | 81 | 107 | 121 | 88 |
| cₛgₛcₛcₛcₛ B | 27030 | 1227 | H02 | 2 | 89 | 93 | 89 |
| cₛgₛcₛcₛuₛ B | 27032 | 1227 | H04 | 76 | 88 | 98 | 90 |
| cₛgₛcₛgₛcₛ B | 27034 | 1227 | H06 | 82 | 91 | 96 | 91 |
| cₛgₛgₛuₛuₛ B | 27040 | 1227 | H12 | 82 | 88 | 97 | 92 |
| cₛgₛgₛaₛaₛ B | 27041 | 1228 | A01 | 0 | 92 | 79 | 93 |
| cₛgₛgₛaₛcₛ B | 27042 | 1228 | A02 | 80 | 107 | 106 | 94 |
| cₛgₛgₛaₛgₛ B | 27043 | 1228 | A03 | 72 | 96 | 84 | 95 |
| cₛgₛgₛcₛaₛ B | 27045 | 1228 | A05 | 80 | 92 | 83 | 96 |
| cₛgₛgₛgₛaₛ B | 27049 | 1228 | A09 | 72 | 87 | 81 | 97 |
| cₛgₛgₛgₛgₛ B | 27051 | 1228 | A11 | 72 | 94 | 86 | 99 |
| cₛgₛuₛgₛcₛ B | 27066 | 1228 | C02 | 81 | 110 | 115 | 101 |
| cₛuₛcₛaₛaₛ B | 27089 | 1228 | E01 | 78 | 106 | 109 | 103 |
| cₛuₛcₛaₛcₛ B | 27090 | 1228 | E02 | 78 | 104 | 117 | 104 |
| cₛuₛcₛuₛcₛ B | 27102 | 1228 | F02 | 74 | 115 | 121 | 105 |
| cₛuₛcₛuₛgₛ B | 27103 | 1228 | F03 | 82 | 107 | 116 | 106 |
| cₛuₛgₛgₛcₛ B | 27114 | 1228 | G02 | 70 | 113 | 118 | 108 |
| cₛuₛuₛcₛaₛ B | 27125 | 1228 | H01 | 68 | 98 | 93 | 110 |
| cₛuₛuₛcₛcₛ B | 27126 | 1228 | H02 | 0 | 92 | 79 | 111 |
| cₛuₛuₛcₛuₛ B | 27128 | 1228 | H04 | 69 | 95 | 81 | 112 |
| cₛuₛuₛgₛcₛ B | 27130 | 1228 | H06 | 76 | 94 | 80 | 113 |
| cₛuₛuₛgₛuₛ B | 27132 | 1228 | H08 | 67 | 94 | 84 | 114 |
| cₛuₛuₛuₛcₛ B | 27134 | 1228 | H10 | 70 | 90 | 83 | 115 |
| cₛuₛuₛuₛuₛ B | 27136 | 1228 | H12 | 70 | 90 | 84 | 116 |
| gₛaₛgₛgₛaₛ B | 27177 | 1229 | D05 | 84 | 97 | 118 | 117 |
| gₛuₛcₛuₛ B | 26456 | 1234 | G03 | 84 | 99 | 115 | 17 |
| gₛuₛuₛcₛ B | 26462 | 1234 | G06 | 80 | 92 | 115 | 18 |
| gₛgₛaₛuₛgₛ B | 27279 | 1230 | D11 | 112 | 96 | 75 | 124 |
| gₛgₛcₛuₛgₛ B | 27295 | 1230 | F03 | 84 | 92 | 103 | 125 |
| gₛgₛgₛgₛuₛ B | 27308 | 1230 | G04 | 81 | 103 | 89 | 137 |
| gₛgₛgₛuₛuₛ B | 27312 | 1230 | G08 | 82 | 100 | 99 | 141 |
| gₛgₛuₛgₛuₛ B | 27324 | 1230 | H08 | 72 | 103 | 97 | 145 |
| uₛcₛaₛcₛcₛ B | 27462 | 1232 | D02 | 84 | 96 | 103 | 160 |
| uₛcₛcₛaₛcₛ B | 27474 | 1232 | E02 | 82 | 96 | 103 | 161 |
| uₛcₛcₛuₛaₛ B | 27485 | 1232 | F01 | 81 | 97 | 100 | 162 |
| uₛcₛcₛuₛcₛ B | 27486 | 1232 | F02 | 77 | 95 | 101 | 163 |
| uₛcₛgₛgₛaₛ B | 27497 | 1232 | G01 | 83 | 102 | 104 | 164 |
| uₛcₛuₛcₛuₛ B | 27512 | 1232 | H04 | 83 | 104 | 95 | 167 |
| uₛcₛuₛgₛuₛ B | 27516 | 1232 | H08 | 83 | 104 | 94 | 168 |
| uₛcₛuₛuₛuₛ B | 27520 | 1232 | H12 | 80 | 104 | 101 | 169 |
| uₛgₛuₛgₛgₛ B | 27579 | 1233 | E11 | 116 | 94 | 84 | 177 |
| uₛuₛcₛcₛuₛ B | 27608 | 1233 | H04 | 79 | 105 | 104 | 180 |
| uₛuₛcₛgₛgₛ B | 27611 | 1233 | H07 | 107 | 102 | 84 | 181 |
| cₛgₛaₛaₛ B | 26369 | 1221 | A01 | 83 | 97 | 94 | 10 |
| uₛaₛaₛaₛ B | 26561 | 1223 | A01 | 74 | 93 | 89 | 37 |
| uₛaₛuₛuₛ B | 26576 | 1223 | B08 | 104 | 96 | 81 | 38 |
| aₛaₛaₛcₛcₛ B | 26630 | 1223 | B10 | 109 | 98 | 84 | 40 |
| aₛcₛgₛgₛaₛ B | 26729 | 1224 | G01 | 76 | 103 | 107 | 42 |
| aₛcₛuₛcₛcₛ B | 26742 | 1224 | H02 | 78 | 103 | 101 | 44 |
| aₛgₛaₛaₛaₛ B | 26753 | 1225 | A01 | 74 | 95 | 81 | 45 |
| aₛgₛaₛaₛgₛ B | 26755 | 1225 | A03 | 75 | 85 | 93 | 46 |
| aₛgₛaₛcₛaₛ B | 26757 | 1225 | A05 | 82 | 94 | 82 | 47 |
| aₛgₛaₛcₛgₛ B | 26759 | 1225 | A07 | 84 | 91 | 82 | 48 |
| aₛgₛaₛgₛgₛ B | 26763 | 1225 | A11 | 75 | 106 | 90 | 49 |
| aₛgₛcₛgₛcₛ B | 26778 | 1225 | C02 | 78 | 103 | 112 | 54 |
| aₛuₛcₛcₛcₛ B | 26838 | 1225 | H02 | 86 | 98 | 79 | 63 |
| aₛuₛcₛcₛuₛ B | 26840 | 1225 | H04 | 89 | 101 | 83 | 64 |
| aₛuₛcₛgₛcₛ B | 26842 | 1225 | H06 | 94 | 98 | 82 | 65 |
| aₛuₛcₛuₛcₛ B | 26846 | 1225 | H10 | 92 | 96 | 82 | 66 |
| aₛuₛcₛuₛuₛ B | 26848 | 1225 | H12 | 80 | 105 | 81 | 67 |
| cₛaₛuₛcₛcₛ B | 26934 | 1226 | H02 | 76 | 104 | 100 | 76 |

a = 2′-O-allyl adenosine
g = 2′-O-allyl guanosine
u = 2′-O-allyl undine
c = 2′-O-allyl cytidine
B = inverted deoxyabasic
S = phosphorothioate internucleotide linkage

TABLE IV

| | | | | | |
|---|---|---|---|---|---|
| Reagent | Equivalents | Amount | Wait Time* DNA | Wait Time* 2′-O-methyl | Wait Time* RNA |

A. 2.5 μmol Synthesis Cycle ABI 394 Instrument

| | | | | | |
|---|---|---|---|---|---|
| Phosphoramidites | 6.5 | 163 μL | 45 sec | 2.5 min | 7.5 min |
| S-Ethyl Tetrazole | 23.8 | 238 μL | 45 sec | 2.5 min | 7.5 min |
| Acetic Anhydride | 100 | 233 μL | 5 sec | 5 sec | 5 sec |
| N-Methyl | 186 | 233 μL | 5 sec | 5 sec | 5 sec |
| Imidazole | | | | | |
| TCA | 176 | 2.3 mL | 21 sec | 21 sec | 21 sec |
| Iodine | 11.2 | 1.7 mL | 45 sec | 45 sec | 45 sec |
| Beaucage | 12.9 | 645 μL | 100 sec | 300 sec | 300 sec |
| Acetonitrile | NA | 6.67 mL | NA | NA | NA |

B. 0.2 μmol Synthesis Cycle ABI 394 Instrument

| | | | | | |
|---|---|---|---|---|---|
| Phosphoramidites | 15 | 31 μL | 45 sec | 233 sec | 465 sec |
| S-Ethyl Tetrazole | 38.7 | 31 μL | 45 sec | 233 min | 465 sec |
| Acetic Anhydride | 655 | 124 μL | 5 sec | 5 sec | 5 sec |
| N-Methyl | 1245 | 124 μL | 5 sec | 5 sec | 5 sec |
| Imidazole | | | | | |
| TCA | 700 | 732 μL | 10 sec | 10 sec | 10 sec |
| Iodine | 20.6 | 244 μL | 15 sec | 15 sec | 15 sec |
| Beaucage | 7.7 | 232 μL | 100 sec | 300 sec | 300 sec |
| Acetonitrile | NA | 2.64 mL | NA | NA | NA |

C. 0.2 μmol Synthesis Cycle 96 well Instrument

| | Equivalents: DNA/ | Amount: DNA/2′-O- | | Wait Time* 2′-O- | |
|---|---|---|---|---|---|
| Reagent | 2′-O-methyl/Ribo | methyl/Ribo | Wait Time* DNA | methyl | Wait Time* Ribo |
| Phosphoramidites | 22/33/66 | 40/60/120 μL | 60 sec | 180 sec | 360 sec |
| S-Ethyl Tetrazole | 70/105/210 | 40/60/120 μL | 60 sec | 180 min | 360 sec |
| Acetic Anhydride | 265/265/265 | 50/50/50 μL | 10 sec | 10 sec | 10 sec |
| N-Methyl | 502/502/502 | 50/50/50 μL | 10 sec | 10 sec | 10 sec |
| Imidazole | | | | | |
| TCA | 238/475/475 | 250/500/500 μL | 15 sec | 15 sec | 15 sec |
| Iodine | 6.8/6.8/6.8 | 80/80/80 μL | 30 sec | 30 sec | 30 sec |
| Beaucage | 34/51/51 | 80/120/120 | 100 sec | 200 sec | 200 sec |
| Acetonitrile | NA | 1150/1150/1150 μL | NA | NA | NA |

*Wait time does not include contact time during delivery.
*Tandem synthesis utilizes double coupling of linker molecule

1. A small-mer molecule having anti-HIV activity, wherein said small-mer comprises about 3 to about 6 nucleotides.

2. A small-mer molecule having anti-proliferative activity, wherein said small-mer comprises about 3 to about 6 nucleotides.

3. The small-mer molecule of claim 1, wherein said small-mer molecule is adapted for use to treat HIV infection.

4. The small-mer molecule of claim 2, wherein said small-mer molecule is adapted for use to treat cancer.

5. A small-mer molecule having any of SEQ ID NOs. 1-182.

6. The small-mer molecule of claim 1, wherein said small-mer molecule comprises one or more internucleotide linkages having Formula I: 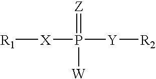
wherein each R1 and R2 is independently any nucleotide, non-nucleotide, or small-mer which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all 0.

7. The small-mer molecule of claim 2, wherein said small-mer molecule comprises one or more internucleotide linkages having Formula I: 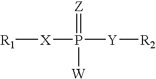
wherein each R1 and R2 is independently any nucleotide, non-nucleotide, or small-mer which can be naturally occurring or chemically modified, each X and Y is independently O, S, N, alkyl, or substituted alkyl, each Z and W is independently O, S, N, alkyl, substituted alkyl, O-alkyl, S-alkyl, alkaryl, or aralkyl, and wherein W, X, Y and Z are not all O.

8. The small-mer molecule of claim 1, wherein said small-mer comprises one or more nucleotides or non-nucleotides having Formula II: 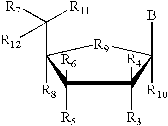
wherein each R3, R4, R5, R6, R7, R8, R10, R 11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base or any other non-naturally occurring base or a non-nucleosidic base or any other non-naturally occurring universal base.

9. The small-mer molecule of claim 2, wherein said small-mer comprises one or more nucleotides or non-nucleotides having Formula II: 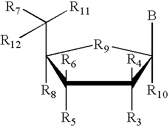
wherein each R3, R4, R5, R6, R7, R8, R10, R 11 and R12 is independently H, OH, alkyl, substituted alkyl, alkaryl or aralkyl, F, Cl, Br, CN, CF3, OCF3, OCN, O-alkyl, S-alkyl, N-alkyl, O-alkenyl, S-alkenyl, N-alkenyl, SO-alkyl, alkyl-OSH, alkyl-OH, O-alkyl-OH, O-alkyl-SH, S-alkyl-OH, S-alkyl-SH, alkyl-S-alkyl, alkyl-O-alkyl, ONO2, NO₂, N3, NH2, aminoalkyl, aminoacid, aminoacyl, ONH2, O-aminoalkyl, O-aminoacid, O-aminoacyl, heterocycloalkyl, heterocycloalkaryl, aminoalkylamino, polyalklylamino, substituted silyl, or group having Formula I; R9 is O, S, CH2, S═O, CHF, or CF2, and B is a nucleosidic base or any other non-naturally occurring base or a non-nucleosidic base or any other non-naturally occurring universal base.

10. A composition comprising the small-mer of claim 1 and a pharmaceutically acceptable carrier or diluent.

11. A composition comprising the small-mer of claim 2 and a pharmaceutically acceptable carrier or diluent.

12. A method for generating a small-mer with improved antiviral activity comprising:
(a) providing the small-mer of claim 1 as a scaffold for additional nucleotides and/or non-nucleotides, (b) generating a library of small-mers by extending the length of the scaffold using all possible nucleotide and/or non-nucleotide combinations for a fixed additional small-mer length, and (c) assaying the small-mer molecule of (b) under conditions suitable for isolating a small-mer having improved antiviral activity.

13. A method generating a small-mer with improved antiproliferative activity comprising:
(a) providing the small-mer of claim 2 as a scaffold for additional nucleotides and/or non-nucleotides, (b) generating a library of small-mers by extending the length of the scaffold using all possible nucleotide and/or non-nucleotide combinations for a fixed additional small-mer length, and (c) assaying the small-mer molecule of (b) under conditions suitable for isolating a small-mer having improved antiproliferative activity.

14. The method of claim 12, wherein the fixed additional length is about 1 to about 10 additional nucleotides and/or non-nucleotides.

15. The method of claim 13, wherein the fixed additional length is about 1 to about 10 additional nucleotides and/or non-nucleotides.

16. The small-mer of claim 1 or claim 2 comprising a 5′-cap, 3′-cap, or a 5′ and 3′-cap moiety.

17. The small-mer of claim 1 or claim 2 comprising one or more abasic moiety.

18. The small-mer of claim 16, wherein said 5′-cap is an inverted abasic moiety.

19. The small-mer of claim 16, wherein said 3′-cap is an inverted abasic moiety.

20. The small-mer of claim 16, wherein said 5′-cap and said 3′-cap is an inverted abasic moiety.
